(19) United States
(12) Patent Application Publication
(10) Pub. No.: US 2008/0255232 A1
(43) Pub. Date: Oct. 16, 2008

(54) Naphthyl Derivatives as Inhibitors of Beta-Amyloid Aggregation

(75) Inventors: Patrizia Minetti, Rome (IT); Roberto Di Santo, Rome (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Rome (IT)

(21) Appl. No.: 12/090,033

(22) PCT Filed: Oct. 12, 2006
(86) PCT No.: PCT/EP2006/067323

§ 371 Date: Apr. 11, 2008

(30) Foreign Application Priority Data
Oct. 18, 2005 (EP) 05022706.5

(51) Int. Cl. A61K 31/245; C07C 211/55; A61K 31/136; A61K 31/10; C07C 317/24; C07C 229/60

(52) U.S. Cl. 514/535; 564/428; 514/657; 560/48; 568/29; 514/709

(57) ABSTRACT

Compounds useful in the treatment of disorders characterized by deposits of amyloid aggregates are herein described together with pharmaceutical compounds containing the same. In particular the compounds of the present invention are those having the Formula (I) as reported below, where the radicals have the meaning indicated in the description.

### FIELD OF THE INVENTION

The present invention relates to new compounds useful in the treatment of disorders characterised by deposits of amyloid aggregates, as well as to the pharmaceutical compounds containing the same together with pharmaceutically acceptable excipients.

### BACKGROUND OF THE INVENTION

The presence of amyloid deposits and changes in the neuronal cytoskeleton are among the clearest signs of Alzheimer's disease (AD). These two events, which involve mainly the cerebral cortex at an early stage, even if the final pathological picture of the disease involves the whole central nervous system, are a necessary, even if not a sufficient, condition for the onset of the disease (Chen M. (1998) Frontiers in Bioscience 3a, 32-37).

In general, irrespective of the protein from which it is formed, the amyloid substance has the characteristics of consisting of fibres 7-8 nm in diameter, of having an affinity for the Congo Red stain and of not being soluble in water. In AD, the amyloid fibres accumulate outside the cell, in the intracellular spaces of the brain and in the tunica media of the cortical and meningeal arterioles, producing three different macroscopic changes: senile plaques and diffuse plaques, which can be differentiated between in that there is the presence or absence of a change in the neuronal processes around the central amyloid deposit, and amyloid angiopathy, which is the expression of the infiltration of amyloid fibres in the wall of the arteries, between the smooth muscle fibres and the internal elastic lamina.

Apart from the formation of amyloid and helical filaments, a very serious synaptic rarefaction has been found in the cortex of subjects suffering from AD. Approximately 80%-90% of the neuronal contacts are destroyed in the final stage of the disease and this change is the real pathological correlate of dementia. Analysing the progress of dementia, it appears certain that amyloid is the early and primary change in the disease and that the intraneuronal helical filaments are the intermediate expression of the damage to the neurons which, ultimately, lose the synaptic contacts, with the subsequent clinical effect of the deterioration in mental functions.

The soluble form of a particular type of β-amyloid, βA₁₋₄₂, hitherto considered to be toxic only in its aggregated form, is implicated in the progressive loss of memory and of the cognitive functions of Alzheimer's patients. βA₁₋₄₂, produced in the initial stage of the disease, suppresses the activity of pyruvate dehydrogenase which promotes the synthesis of ACh providing for the transportation of acetyl-CoA, reducing the release of the neurotransmitter, changing the synaptic connections and causing the cholinergic deficits responsible for the disease (Hoshi M., Takashima A., Murayama M., Yasutake K., Yoshida N., Ishiguro K., Hoshino T., Imahori K. (1997) The Journal of Biological Chemistry 272:4, 2038-2041).

It is known that a number of the stains bind to the amyloid fibres in a specific way and the most important of these is Congo Red (CR) (Lorenzo A. and Yankner B. A, 1994 PNAS 91; 12243-12247).

This stain causes an increase in birefringence of the amyloid fibres and produces a characteristic circular dichroism indicative of a specific interaction between the stain and the substrate (the fibres) enabling diagnosis of amyloidosis in the tissue.

The protein β-amyloid (βA) derives from the proteolytic action of a number of enzymes which act specifically on the precursor of the amyloid protein (βAPP) (Vassar R. et al. 1999 Science 286; 735-740).

There are many mechanisms by which the β-amyloid fragment can induce neurotoxic effects. In the first place immunohistochemical studies have revealed the presence, in the senile plaques, of inflammation interleukins (IL-1, IL-6), complement factors, other inflammatory factors and lysosomial hydrolases. It has been demonstrated that the β-amyloid protein is capable of stimulating the synthesis and secretion of IL-1, IL-6 and IL-8 by the microglial cells and therefore of activating the cytotoxic mechanisms of acute inflammation (Sabbagh M. N., Galasko D., Thal J. L. (1997) Alzheimer's Disease Review 3, 1-19). The presence of activated microglia in postmortem Alzheimer disease specimens is used to support the argument that inflammation contributes to Alzheimer pathogenesis (Morgan D. et al, (2005) J. Neuropathol Exp. Neurol 64(9):743-753)

Diseases characterised by deposits of amyloid aggregates include, apart from Alzheimer's disease, Down's syndrome, hereditary cerebral haemorrhage associated with amyloidosis of the “Dutch type”, amyloidosis accompanied by chronic inflammation, amyloidosis accompanied by multiple myeloma and other dyscrasias of the haematic “B” lymphoid cells, amyloidosis accompanied by type II diabetes, amyloidosis accompanied by prion diseases such as Creutzfeldt-Jakob disease and Gerstmann-Straussler syndrome, kuru and ovine scrapie.

In general, however, the damage caused by βA may be summarised as follows:

1. changes in amyloidogenesis;

2. increase in the vulnerability of neurons to excitotoxicity;

3. increase in the vulnerability of the neurons to hypoglycaemic damage;

4. changes in the homeostasis of calcium;

5. increase in damage by oxidation;

6. activation of the inflammatory mechanisms;

7. activation of the microglia;

8. induction of lysosomial proteases;

9. changes in the phosphorylation of the protein tau;

10. induction of apoptosis;

11. damage to the membranes.

From a purely theoretical point of view, the reduction in the damage caused by βA can be dealt with by different therapeutic approaches:
a) reducing the production of βA using inhibitors of the secretases to change the metabolism of the APP (increasing the α or reducing the β and γ secretases);
b) preventing or blocking the aggregation of the βA;
c) increasing the clearance of the βA;
d) blocking the neurotoxic effects of βA restoring calcium homeostasis;
e) preventing the toxicity produced by the free radicals;
f) preventing excitotoxicity;
g) reducing the damage caused by the inflammatory response;
h) correcting the imbalance between zinc and copper;
i) inhibiting neuronal apoptosis
   (Sabbagh M. N., Galasko D., That L. J. (2000) Alzheimer's Disease Review 3-4, 231-59; Rogers J. Y. and Lahiri D. K. (2004) Curr Drug Targets 6:535-551; Jacobsen J. S. (2002) Curr. Top Med Chem (2002) 4:343-52; Dodel R. C., Hampel H., Du Y. (2003) Lancet Neurol 4:215-20).

To date no specific therapy exists to prevent, slow down or arrest the amyloidogenic process at the root of Alzheimer's disease.

Indeed the treatments currently used for this disease are exclusively symptomatic and, even if they act on various aspects, they fundamentally only interfere with the neurotransmitter mechanisms which govern learning and memory. The substances mostly used include the reversible inhibitors of acetylcholinesterase, such as tacrine, donepezil and rivastigmine.

Furthermore, the only diagnostic tools currently available to diagnose Alzheimer's disease are behavioural examinations and clinical “scores”, while, due to an absence of suitable tracers, radiographic or scanning procedures are not yet able to accurately distinguish between degeneration of an Alzheimer's type and other degenerative phenomena.

The problems encountered in treating Alzheimer's disease, the severity of this disease and the difficulty of diagnosing it, make it desirable to not only find new drugs which are able to cure or slow down the progress of the disease but also discover compounds to be used in radiographic and scanning procedures capable of diagnosing it.

The Applicant had earlier discovered (WO02/00603) that pamoic acid, or one of its derivatives, or one of its analogues, or one of their pharmaceutically acceptable salts, are effective in the treatment and in the prevention of Alzheimer's disease and diseases characterised by deposits of amyloid aggregates.

Published patent application US 2004/0229869 discloses and claims mercatophenyl naphthyl methane compounds, which are said to be potentially useful in the treatment of osteoporosis.

Published patent application US 2005/0119225 discloses and claims N-substituted aniline and diphenylamine analogs, which are said to be PDE4 inhibitors.

Published patent application US 2004/0053890 relates to naphthalene derivatives whose biological activity would be linked to the cannabinoid receptor, thus potentially useful in the treatment of pain and inflammation. These compounds are defined by a general formula, according to which the naphthyl group always brings two substituents in positions 1 and 4. Focussing on the synthesized compounds, those in which the substituent in position 1 is NH, S or SO₂ (see Table at page 6) in position 4 always present the radical pentyl-oxy.

German patent DE 343057 claims the synthesis of 1-arylamino-4-oxynaphthalines.

Published patent application US 2004/0132769 relates to phenylacetic acid derivatives reported to have an activity as selective COX-2 inhibitors.

Moosmann et al. (see Biol. Chem., 382., 1601-12, 2001) report the protective activity of some aromatic amines and imines against oxidative nerve cell death. According to this study the compounds, which showed superior effects among those tested in the antioxidant neuroprotection, were iminostilbene, phenoxazine and phenothiazine and in general imines were shown to be more potent than the corresponding amines.

The blood brain barrier crossing always represents one the main problems for all the compounds acting on the CNS. Therefore there is always the need of discovering compounds that, while maintaining or improving the efficacy in all the in-vitro tests, are also able to cross the blood brain barrier.

### DESCRIPTION OF THE INVENTION

The Applicant has now surprisingly found new compounds which are effective in the treatment of the diseases referred to. These compounds tested on animals have also shown the capability to cross the blood brain barrier. These results are reported in the section entitled Examples.

The Applicant has also found that some compounds, whose structure and synthesis has already been reported, show unexpectedly interesting pharmacological activity in the same field.

One of the main objects of the present invention is the use of the compounds of Formula (I) as follows, for the preparation of pharmaceutical compounds useful in the treatment of conditions characterised by deposits of amyloid aggregates.

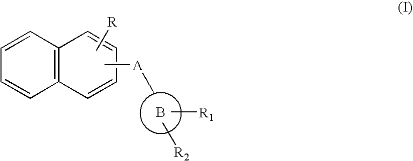

where:
R is selected from the group consisting of H, OR₃, COOR₃, N(R₃)₂, NO₂, halogen, hydroxyalkyl C₁-C₃;
R₁ and R₂ are the same or different and are selected from the group consisting of H; OR₃; COOR₃; linear or branched, saturated or unsaturated C₁-C₄ alkyl; N(R₃)₂; C₁-C₄ linear or branched, saturated or unsaturated alkylthio; halogen; and SO₂N(R₃)₂;
R₃ is selected from the group consisting of H; C₁-C₄ linear or branched alkyl; PO₃H₂; and PO₃(CH₃)₂;
A is selected from the group consisting of NR₄; S; and SO₂;
R₄ is selected from the group consisting of H; C₁-C₄ linear or branched alkyl; C₁-C₄ linear or branched alkanoyl; and
B is a phenyl or naphthyl group.
According to independently preferred embodiments of the invention A is NH, R₁ is H, R₂ is selected from the group consisting of H, COOH, COOCH₃ and OH; and R is selected from the group consisting of H, OH and OCH₃.

The following Table 1 lists some of the compounds, together with their structural formula, whose use according to the invention is preferred.

TABLE 1

| ID No. | Name | Structure |
|---|---|---|
| ST2762 | 1-hydroxy-N-phenylnaphthalen-2-aminiumchloride | 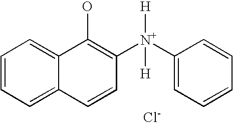 |
| ST2763 | methyl 4-(1-naphthylamino)benzoate | 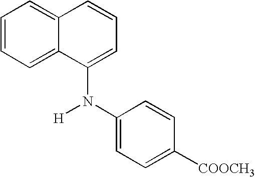 |
| ST2764 | 4-(1-naphthylamino)benzoic acid | 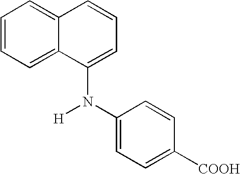 |
| ST2177 | 4-(4-hydroxyanilino)-1-naphthol | 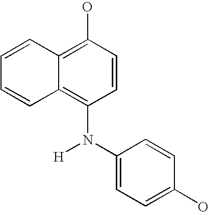 |
| ST2176 | 4-anilino-1-naphthol | 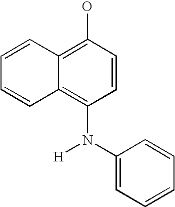 |
| ST2757 | 2-[(2-hydroxy-1-naphthyl)amino]benzoic acid | 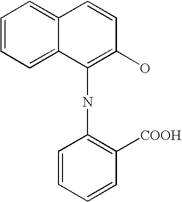 |
| ST2756 | (1-methoxy-2-naphthyl)phenylamine | 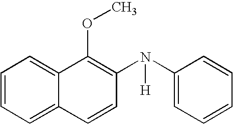 |
| ST2173 | 4-methoxy-N-phenyl-1-naphthalenamine | 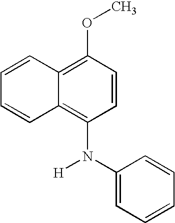 |
| ST3499 | 1-methoxy-4-[(4-methoxyphenyl)sulfonyl]naphthalene | 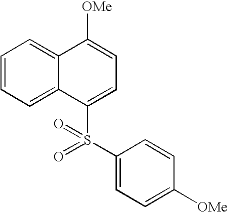 |
| ST3500 | 4-[(4-hydroxyphenyl)sulfonyl]-1-naphthol | 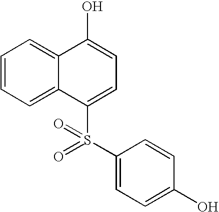 |

Another object of the present invention are the compounds of general Formula (I)

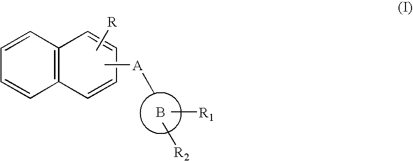

where:
R is selected from the group consisting of H, OR₃, COOR₃, N(R₃)₂, NO₂, halogen, hydroxyalkyl C₁-C₃;
R₁ and R₂ are the same or different and are selected from the group consisting of H; OR₃; COOR₃; linear or branched, saturated or unsaturated C₁-C₄ alkyl; N(R₃)₂; C₁-C₄ linear or branched, saturated or unsaturated alkylthio; halogen; and SO₂N(R₃)₂; provided that R₁ and R₂ are not both H or halogen;
R₃ is selected from the group consisting of H; C₁-C₄ linear or branched alkyl; PO₃H₂; and PO₃(CH₃)₂;
A is selected from the group consisting of NR₄; S; and SO₂;
R₄ is selected from the group consisting of H; C₁-C₄ linear or branched alkyl; C₁-C₄ linear or branched alkanoyl; and
B is a phenyl or naphthyl group,
with the proviso that:
when A is NR₄, R₁ and R₂ are not both OR₃; and
with the exception of the following compounds:
4-methoxy-N-phenyl-1-naphthalenamine (ST2173),
1-hydroxy-N-phenylnaphthalen-2-aminium chloride (ST2762),
methyl 4-(1-naphthylamino)benzoate (ST2763),
4-(1-naphthylamino)benzoic acid (ST2764),
4-(4-hydroxyanilino)-1-naphthol (ST2177),
4-anilino-1-naphthol (ST2176),
2-[(2-hydroxy-1-naphthyl)amino]benzoic acid (ST2757),
(1-methoxy-2-naphthyl)phenylamine (ST2756);
1-methoxy-4-[(4-methoxyphenyl)sulfonyl]naphthalene (ST3499); and
4-[(4-hydroxyphenyl)sulfonyl]-1-naphthol (ST3500).

As a matter of fact the synthesis of all the compounds listed here above has been mentioned in previous publications, specifically as follows:

ST2756: Bowman, D. F.; Middleton, B. S.; Ingold, K. U. Oxidation of amines with peroxy radicals. I. N-phenyl-2-naphthylamine. Journal of Organic Chemistry (1969), 34(11), 3456-61;
ST2763: Seki, Mieko; Yoneyama, Hiroto; Okuda, Daisuke; Hirose, Eiichi; Ozaki, Tadayoshi; Agata, Takashi; Ishii, Toru; Mashimo, Kiyokazu; Sato, Katsuhiro. Electric charge-transportable polymers with high glass transition temperature, good solvent solubility, film-forming property and thermal stability. Jpn. Kokai Tokkyo Koho (2003), 34 pp;
ST2764: Wagner, Eugene Ross; Allen, Bobbie Jewel; Renzi, Alfred Arthur. p-Aminobenzoic acids with hypolipemic action. Ger. Offen. (1977), 13 pp;
ST2757: Mehta, R. K.; Gupta, R. K.; Singhi, V. C. Uranium(VI) complexes of some tridentate Schiff bases. Israel Journal of Chemistry (1971), 9(5), 589-91 and Ozha, D. D.; Mehta, R. K. Stepwise formation and thermodynamic constants of europium, gadolinium, dysprosium and holmium complexes of some tridentate Schiff bases. Transactions of the SAEST (1979), 14(3), 141-4;
ST2176: Hotta, Seiji; Ito, Yukiaki; Hatori, Minoru. Fluoran derivatives. Jpn. Kokai Tokkyo Koho (1975), 18 pp; and Yuan, Xin-hua; Xu, Hong-xing; Ni, Zhong-hai; Zhang, Li-fang; Wei-Xian-yong. Study on the reaction of aromatics containing active hydrogen atom with nitrobenzene catalyzed by aluminum trichloride. Ranliao Huaxue Xuebao (2004), 32(1), 104-108;
ST2173: Justus Liebigs Ann. Chem. (1925) 443, 222; ST2762: Bull. soc. chim. (1925), 37, 890-901; ST3499: U.S. Pat. No. 4,996,279-U.S. Pat. No. 4,960,912; and ST3500: U.S. Pat. No. 4,996,279-U.S. Pat. No. 4,960,912.

The present invention also comprises tautomers, geometrical isomers, optically active forms as enantiomers, diastereomers and racemate forms, as well as pharmaceutically acceptable salts of the compounds of Formula (I).

Preferred pharmaceutically acceptable salts of the Formula (I) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

According to independently preferred embodiments of the invention: A is NH, R is selected between OH and OCH₃ and/or is present on the naphthyl group in ortho position with respect to A, R₁ is selected among OCH₃, COOCH₃, H, COOH and R₂ is selected among H, I, OH and OCH₃.

Within the framework of the present invention, examples of linear or branched C₁-C₄ alkyl group, are understood to include methyl, ethyl, propyl, butyl, and their possible isomers, such as, for example, isopropyl, isobutyl and ter-butyl.

The following Table 2 lists some of the most preferred compounds according to the invention together with their structural formula.

TABLE 2

| ID No. | Name | Structure |
|---|---|---|
| ST2759 | methyl 2-[(2-hydroxy-1-naphthyl)amino]benzoate | 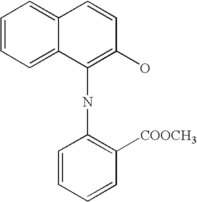 |
| ST2760 | methyl 2-[(2-methoxy-1-naphthyl)amino]benzoate | 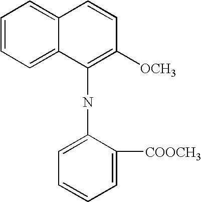 |
| ST1972 | 4-[(4-methoxy-1-naphthyl)amino]benzoic acid | 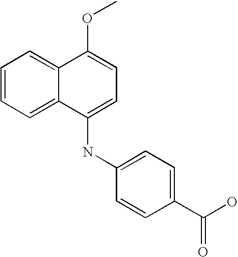 |
| ST1973 | 4-[(4-hydroxy-1-naphthyl)amino]benzoic acid | 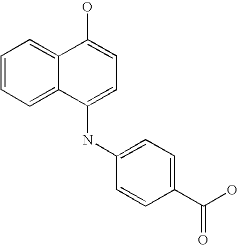 |
| ST2878 | N-(5-iodo-2-methoxyphenyl)-N-(4-methoxy-1-naphthyl)amine | 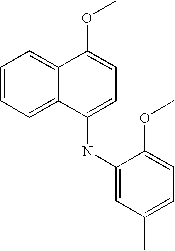 |
| ST2879 | N-(4-methoxy-1-naphthyl)-N-(2-methoxyphenyl)amine | 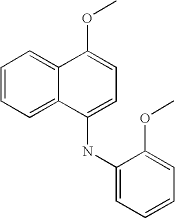 |
| ST2761 | 2-methoxy-N-(2-methoxy-1-naphthyl)naphthalen-1-amine | 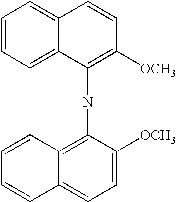 |
| ST2178 | methyl 4-[(4-hydroxy-1-naphthyl)amino]benzoate | 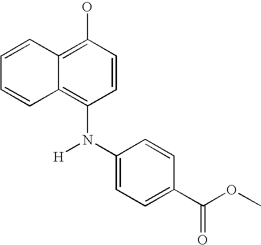 |
| ST2511 | 2-hydroxy-5-[(4-hydroxy-1-naphthyl)amino]benzoicacid hydrochloride | 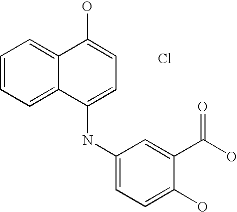 |
| ST2174 | 2-methoxy-5-[(4-methoxy-1-naphthyl)amino]benzoic acid | 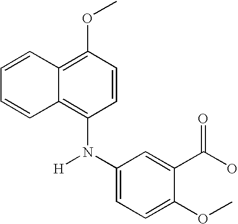 |
| ST2175 | 4-methoxy-N-(4-methoxyphenyl)-1-naphthalenamine | 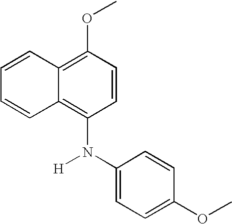 |
| ST3244 | 4-methylbenzoate-1-yl(4-methoxy-1-naphthyl)amine | 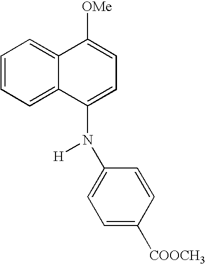 |
| ST3245 | 4-methoxy-3-methylbenzoate-1-yl(4-methoxy-1-naphthyl)amine | 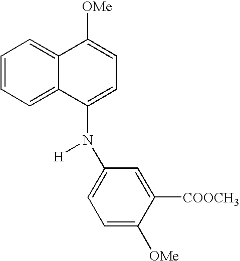 |
| ST3459 | 4-[(1-hydroxy-2-naphthyl)amino]benzoic acid | 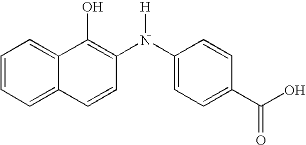 |
| ST3458 | N,N-dimethyl-N′-[4-(methylthio)phenyl]naphthalene-1,4-diamine dihydrochloride | 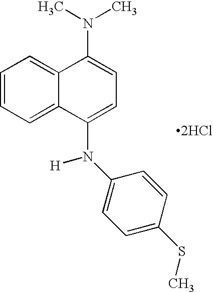 |
| ST3451 | N-(4-methoxyphenyl)-4-nitronaphthalen-1-amine | 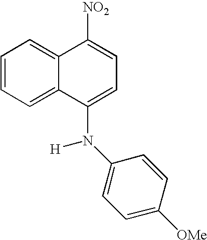 |
| ST3501 | 4-[(4-hydroxyphenyl)thio]-1-naphthol | 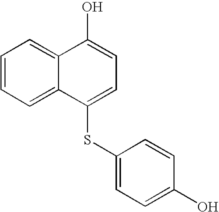 |
| ST3450 | 4-fluoro-N-(4-fluorophenyl)naphthalen-1-aminehydrochloride | 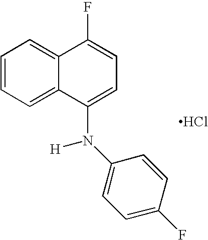 |
| ST3455 | 4-fluoro-N-[4-(methylthio)phenyl]naphthalen-1-amine | 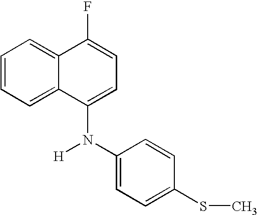 |
| ST3498 | 1-methoxy-4-[(4-methoxyphenyl)thio]naphthalene | 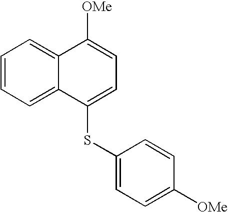 |
| ST3452 | Methyl-4-[(1-methoxy-2-naphthyl)amino]benzoate | 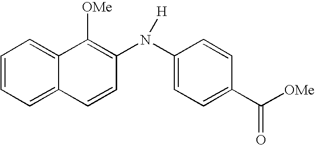 |
| ST3454 | N-(4-iodophenyl)-1-methoxynaphthalen-2-amine | 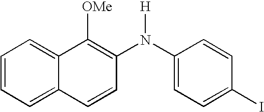 |
| ST3453 | 4-[(1-methoxy-2-naphthyl)amino]benzoicacid | 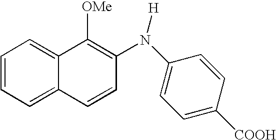 |
| ST3456 | methyl 4-[(1-hydroxy-2-naphthyl)amino]benzoate | 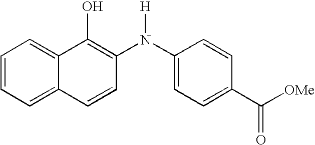 |
| ST3717 | 2-hydroxy-5-[(4-hydroxy-1-naphthyl)amino]benzoic acid | 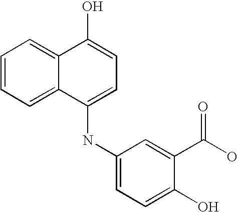 |

Another object of the present invention is the use of the compounds of Formula (I) as medicines, or, in other words, as active principles of drugs, in particular for the treatment of diseases characterised by deposits of amyloid aggregates.

A further object of the present invention is the use of the compounds of Formula (I) referred to above or one of their pharmaceutically acceptable salts, for the preparation of pharmaceutical compositions useful in the treatment of disorders characterised by deposits of amyloid aggregates.

The compounds of Formula (I) may be prepared from readily available starting materials using the following general methods and procedures. It will be appreciated that where typical or preferred experimental conditions (i.e. reaction temperatures, time, moles of reagents, solvents, etc.) are given, other experimental conditions can also be used, unless otherwise stated. Optimum reaction conditions may vary with the particular reactants or solvents used, but such conditions can be determined by one skilled in the art by routine optimisation procedures.

A further object of the present invention is a process for preparing general formula compounds (I). According to preferred embodiments of the invention some of such processes are reported in the section entitled Examples and are diagrammatically represented by some Schemes (see in particular Schemes 1 to 6).

Generally speaking the compounds of Formula (I) may be obtained starting from a substituted or un-substituted nitro naphthalene. The nitro naphthalene is hydrogenated with catalyst such as Pd/C in organic solvent such as ethyl acetate. The amine so obtained is condensed with a substituted or un-substituted aryl halide derivative, with the reagent BINAP [2,2′-Bis(diphenylphosphino)-1,1′-binaphthyl] and Palladium acetate. Next steps are deprotection of ether with BBr₃ and or hydrolysis of ester with NaOH.

A method of treating a mammal suffering from a pathology characterized by deposits of amyloid aggregates, comprising administering a therapeutically effective amount of a compound of Formula (I) as described above represents one of the aspects of the present invention.

The term “therapeutically effective amount” as used herein refers to an amount of a therapeutic agent needed to treat, ameliorate a targeted disease or condition, or to exhibit a detectable therapeutic effect.

For any compound, the therapeutically effective dose can be estimated initially in in vitro assays, for example by measuring the residual aggregated beta-amyloid after incubation with the compounds of the invention; or in animal models, usually mice, rats, rabbits, dogs, pigs or monkeys, such as for example the amyloid precursor protein (APP)-transgenic mice.

The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination (s), reaction sensitivities, and tolerance/response to therapy. This amount can be determined by routine experimentation and is within the judgement of the clinician. Generally, an effective dose will be from 0.01 mg/kg to 100 mg/kg, preferably 0.05 mg/kg to 50 mg/kg. Compositions may be administered individually to a patient or may be administered in combination with other agents, drugs or hormones.

The medicament may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity.

Suitable carriers may be large, slowly metabolised macromolecules such as proteins, polysaccharides, polylactic acids, polyglycolic acids, polymeric amino acids, amino acid copolymers and inactive virus particles.

A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The medicament of this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, intravaginal, rectal means or locally on the diseased tissue after surgical operation.

Dosage treatment may be a single dose schedule or a multiple dose schedule.

A further object of the present invention are pharmaceutical compositions containing one or more of the compounds of formula (I) described earlier, in combination with excipients and/or pharmacologically acceptable diluents.

The compositions in question may, together with the compounds of formula (I), contain other known active principles.

A further embodiment of the invention is a process for the preparation of pharmaceutical compositions characterised by mixing one or more compounds of formula (I) with suitable excipients, stabilizers and/or pharmaceutically acceptable diluents.

A further object of the present invention is the use of the compounds of Formula (I) referred to above, for the preparation of a diagnostic kit for diagnosing conditions characterised by deposits of amyloid aggregates.

Indeed, the compounds according to the present invention may contain in their molecular structure atoms of elements commonly used in diagnostic imaging. For example, radioactive isotopes of carbon, hydrogen, nitrogen, oxygen, iodine and indium can be introduced into their structure. And, more specifically, the compound of formula (I) can have at least one of the elements carbon, hydrogen, nitrogen or oxygen of its own molecular structure replaced by a corresponding radioactive isotope; or carry at least one atom of radioactive iodine; or it is in the form of a complex with radioactive indium.

These compounds containing radioactive isotopes may be prepared by analogy to those previously prepared as reported in the literature.

Zhuang et al. (see Nucl Med Biol. 2005 February; 32(2):171-84) report the preparation of biphenyls labeled with technetium⁹⁹ for imaging beta-amyloid plaques in the brain. Based on previously obtained Amyloid-beta plaque-specific biphenyls containing a p-N,N-dimethylaminophenyl group, a series of ⁹⁹Tc and Re—N₂S₂-biphenyl derivatives was prepared.

Huang Y et al. (see J Med Chem. 2005 Apr. 7; 48(7):2559-70) have worked on fluorinated diaryl sulfides as serotonin transporter ligands. They have reported the synthesis, structure-activity relationship study, and in vivo evaluation of fluorine-18-labeled compounds as PET imaging agents.

A serotonin transporter (SERT) ligand, [¹¹C]2-[2-(dimethylaminomethylphenylthio)]-5-fluorophenylamine was synthesized and evaluated as a candidate PET radioligand in pharmacological and pharmacokinetic studies. As a PET radioligand, AFA can be labeled with either C-11 or F-18 (Huang Y et al., Nucl Med Biol. 2004 August; 31(6):727-38).

All these radioactive compounds are useful for techniques such as PET (Positron Emission Tomography), SPECT (Single Photon Emission Computerized Tomography) and planar scintigraphy. Alternatively, the compounds according to the present invention containing radioactive isotopes or atoms of elements useful as radio-opaque elements (for example iodine), can be used as complexing agents for elements commonly used in diagnostic imaging techniques, such as gadolinium for example (NMR), technetium (scanning techniques).

On the basis of this diagnostic application, the compounds according to the present invention are also useful for the prevention of the diseases indicated above.

The invention will now be illustrated in greater detail by means of non-limiting Examples

### EXAMPLES

### Example 1

### Preparation of Compounds of Formula (I) According to Synthetic

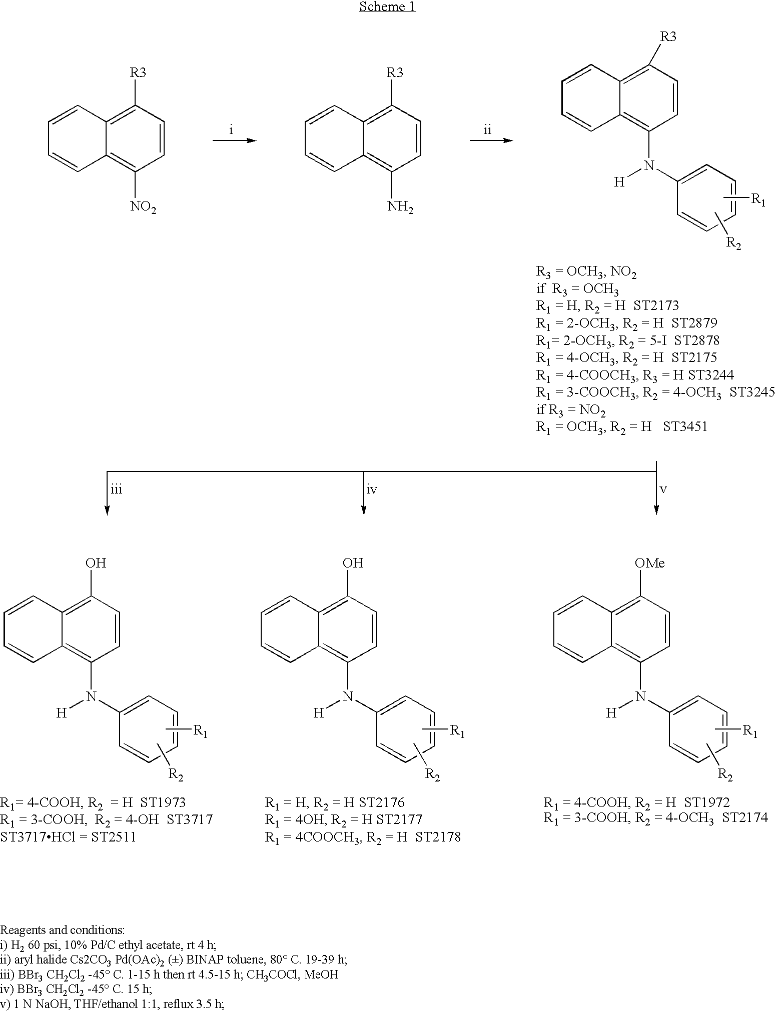

### Step i—Preparation of 4-methoxy-1-naphthalenamine

A suspension of 4-methoxy-1-nitronaphthalene (1.0 g, 4.9 mmol) in ethyl acetate (150 ml) was hydrogenated in Parr apparatus at room temperature in the presence of 10% Pd/C as a catalyst (200 mg) at an initial pressure of 60 psi for 4 h. The catalyst was removed by filtration and the filtrate was dried and evaporated to afford pure 4-metossi-1-naphthalenamine (850 mg, 100% yield), which was used for the next reaction without further purification.

### Step ii—Preparation of 4-methylbenzoate-1-yl(4-methoxy-1-naphthyl)amine (ST3244)

A dried flask was purged with argon and charged with (±) BINAP (70 mg, 0.11 mmol) and capped with a rubber septum. The flask was purged with argon and toluene (9.7 ml) was added. The mixture was heated to 80° C. with stirring until the BINAP dissolved (˜1 min). The solution was cooled to room temperature, the septum was removed, and palladium acetate (16 mg, 0.07 mmol) was added. The flask was recapped with the septum and then purged with argon (for ˜30 sec). The mixture was stirred at room temperature for 1 min, the 4-methoxy-1-naphthalenylamine (600 mg, 3.5 mmol) dissolved in toluene (1.5 ml) and methyl-4-bromobenzoate (615 mg, 2.9 mmol) were added, the septum was removed, and cesium carbonate (1.31 g, 4.0 mmol) was added. Additional toluene (7 ml) was added, then the flask was recapped with the septum, and purged with argon again. The mixture was heated to 80° C. with stirring for 24 h. The mixture was cooled to room temperature, diluted with ether, filtered, and concentrated in vacuo. The crude product (980 mg) was then purified by column chromatography (ethyl acetate/n-hexane 1:1 as eluent) to obtain 820 mg (77%) of pure ST3244. Mp 168-170° C. (benzene); IR: ν 3300 (NH), cm⁻¹; ¹H-NMR (CDCl₃): δ 3.89 (s, 3H, COOCH₃), 4.08 (s, 3H, OCH₃), 6.03 (s broad, 1H, NH), 6.45-6.71 (m, 2H, benzene C3-H and C5-H), 6.85 (d, J=8.1 Hz, naphthalene H), 7.39 (d, J=8.1 Hz, naphthalene H), 7.48-7.61 (m, 2H, naphthalene H), 7.85-7.96 (m, 3H, naphthalene H and benzene C2-H and C6-H), 8.35-8.41 (m, 1H, naphthalene H).

The following compounds were obtained with the same procedure reported above. The reaction time, eluent for chromatographic system, yield, mp (recrystallization solvent), IR, and NMR data are reported for each derivative.

4-methoxy-N-phenyl-1-naphthalenamine (ST2173): 21 h; ethyl acetate/n-hexane 1:1; 70%; mp 141-143° C. (cyclohexane/n-hexane); IR: ν 3400 (NH), cm⁻¹; ¹H-NMR (CDCl₃): δ 4.07 (s, 3H, CH₃), 5.75 (s broad, 1H, NH), 6.75-6.90 (m, 4H, naphthalene H and benzene C2-H and C6-H), 7.15-7.25 (m, 3H, benzene C3-H, C4-H and C5-H), 7.50-7.60 (m, 2H, naphthalene H), 8.04 (m, 1H, naphthalene H), 8.33 (m, 1H, naphthalene H).
4-methoxy-N-(4-methoxyphenyl)-1-naphthalenamine (ST2175): 21 h; ethyl acetate/n-hexane 1:1; 96%; oil; IR: ν 3380 (NH), cm⁻¹; ¹H-NMR (CDCl₃): δ 3.81 and 4.04 (2s, 6H, CH₃), 5.90 (s broad, 1H, NH), 6.74-6.85 (m, 6H, naphthalene C2-H and C3-H and benzene H), 7.51-7.58 (m, 2H, naphthalene H), 8.04 (m, 1H, naphthalene H), 8.35 (m, 1H, naphthalene H).
N-(4-methoxy-1-naphthyl)-N-(2-methoxyphenyl)amine (ST2879): 39 h; ethyl acetate/n-hexane 1:2; 70%; mp 108-110° C. (cyclohexane); IR: ν 3395 cm⁻¹ (NH); ¹H-NMR (CDCl₃): δ 3.98 and 4.02 (2s, 6H, CH₃), 6.60 and 6.91 (2m, 2H, benzene C3-H and C6-H), 6.73 (m, 2H, benzene C4-H and C5-H), 6.80 (d, 1H, Jₒ=8.1 Hz, naphthalene C3-H), 7.35 (d, 1H, Jₒ=8.1 Hz, naphthalene C2-H), 7.48 (m, 2H, naphthalene C6-H and C7-H), 7.98 and 8.30 (2m, 2H, naphthalene C5-H and C8-H).

The following derivatives were obtained using a procedure similar to that reported above. Some reagents were used in a different ratio as explained below.

N-(5-iodo-2-methoxyphenyl)-4-methoxy-1-naphthalenamine (ST2878): the reaction was performed on 1.04 g (6.0 mmol) of 1-methoxy-4-naphthalenamine. 19 h, then BINAP (60 mg, 0.095 mmol), palladium acetate (20 mg, 0.06 mmol), toluene (9 ml); 9 h, then BINAP (120 mg, 0.19 mmol), palladium acetate (30 mg, 0.13 mmol), toluene (18 ml); 24 h, then BINAP (120 mg, 0.19 mmol), palladium acetate (30 mg, 0.13 mmol), toluene (18 ml), 2,4-diiodoanisole (1.8 g, 5.0 mmol); 48 h; flash chromatography, ethyl acetate/n-hexane 1:20; 18%; oil; IR: ν 3390 cm⁻¹ (NH); ¹H-NMR (CDCl₃): δ 3.95 and 4.03 (2s, 6H, CH₃), 6.61 (d, 1H, Jₒ=8.3 Hz, benzene C3-H), 6.78 (d, 1H, Jₘ=2.0 Hz, benzene C6-H), 6.82 (d, 1H, Jₒ=8.1 Hz, naphthalene C3-H), 7.01 (dd, 1H, Jₒ=8.3 Hz, Jₘ=2.0 Hz, benzene C4-H), 7.34 (d, 1H, Jₒ=8.1 Hz, naphthalene C2-H), 7.50 (m, 2H, naphthalene C6-H and C7-H), 7.92 and 8.31 (2m, 2H, naphthalene C5-H and C8-H).

### 4-methoxy-3-methylbenzoate-1-yl(4-methoxy-1-naphthyl)amine (ST3245)

Performed on 4-methoxy-1-naphthalenamine (1.6 g, 9.1 mmol), using (±) BINAP (470 mg, 0.76 mmol), palladium acetate (120 mg, 0.51 mmol) at 120° C. 24 h; ethyl acetate/n-hexane 1:1; 97%; oil; IR: ν 3320 (NH), 1695 (CO) cm⁻¹; ¹H-NMR (CDCl₃): δ 3.88 (s, 3H, OCH₃), 3.89 (s, 3H, OCH₃), 4.04 (s, 3H, OCH₃), 5.60 (s broad, 1H, NH), 6.79 (d, 1H, J=8.2 Hz, naphthalene H), 6.88 (m, 2H, benzene C3-H and C4-H), 7.22 (d, 1H, J=8.2 Hz, naphthalene H), 7.34 (m, 1H, benzene C6-H), 7.50-7.58 (m, 2H, naphthalene H), 7.99 and 8.34 (2m, 2H, naphthalene).

### N-(4-methoxyphenyl)-4-nitronaphthalen-1-amine (ST3451)

A dried flask was purged with argon and charged with (±) BINAP (50 mg, 0.08 mmol) and capped with a rubber septum. The flask was purged with argon and dioxane (7.5 ml) was added. The mixture was heated to 100° C. with stirring until the BINAP dissolved. The solution was cooled to room temperature, the septum was removed, and palladium acetate (13 mg, 0.055 mmol) was added. The flask was recapped with the septum and then purged with argon. The mixture was stirred at room temperature for 1 min, the 1-amino-4-nitro-naphthalene (500 mg, 2.7 mmol) and a solution of 4-bromo-anisole (410 mg, 2.2 mmol) dissolved in dioxane (2 ml) were added, the septum was removed, and cesium carbonate (1.00 g, 3.08 mmol) was added. Additional dioxane (6 ml) was added, then the flask was recapped with the septum, and purged with argon again. The mixture was heated to 100° C. under stirring for 21 h and 15 min. A solution of (±) BINAP (50 mg, 0.08 mmol) and palladium acetate (13 mg, 0.055 mmol) dissolved in dioxane (7.5 ml) was added and stirred at 100° C. for 4 h and 30 min. A solution of (±) BINAP (50 mg, 0.08 mmol) and palladium acetate (13 mg, 0.55 mmol) dissolved in dioxane (7.5 ml) was added and stirred at 100° C. for 3 days. A solution of (±) BINAP (50 mg, 0.08 mmol) and palladium acetate (13 mg, 0.055 mmol) dissolved in dioxane (7.5 ml) was added and stirred at 100° C. for 24 h. A solution of (±) BINAP (50 mg, 0.08 mmol) and palladium acetate (13 mg, 0.055 mmol) dissolved in dioxane (7.5 ml) was added and stirred at 100° C. for 20 h. A solution of (±) BINAP (50 mg, 0.08 mmol) and palladium acetate (13 mg, 0.055 mmol) dissolved in dioxane (7.5 ml) was added and stirred at 100° C. for 24 h. A solution of (±) BINAP (50 mg, 0.08 mmol) and palladium acetate (13 mg, 0.055 mmol) dissolved in dioxane (7.5 ml) was added and stirred at 100° C. for 16 h. A solution of (±) BINAP (50 mg, 0.08 mmol) and palladium acetate (13 mg, 0.055 mmol) dissolved in dioxane (7.5 ml) was added and stirred at 100° C. for 4 h and 30 min. The mixture was cooled to room temperature, diluted with methanol, filtered, and concentrated in vacuo. The crude product (2.86 g) was purified by column chromatography (Chloroform as eluent) to obtain 220 mg (38%) of pure ST3451; IR: ν 3365 (NH), cm⁻¹; ¹H-NMR (DMSO-d₆): δ 3.93 (s, 3H, CH₃), 6.77 (s broad, 1H, NH), 6.85 (m, 1H, naphthalene H), 7.05 (d, 2H, Jₒ=8.8 Hz, benzene C3-H and C5-H), 7.30 (d, 2H, Jₒ=8.8 Hz, benzene C2-H and C6-H), 7.67 and 7.81 (2m, 2H, naphthalene C6-H and C7-H), 8.08, 8.39 and 9.07 (3m, 3H, C2-H, C5-H and C8-H naphthalene).

### Step iii—Preparation of 2-[(2-hydroxy-1-naphthyl)amino]benzoic acid (ST1973)

A solution of methyl 4-(4-methoxy-1-naphthalenylamino)benzoate (ST3244) (763 mg, 2.4 mmol) in dichloromethane (27 ml) was added dropwise to 1M BBr₃ (12.6 ml, 12.6 mmol) in the same solvent at −45° C., under argon atmosphere. The mixture was stirred for 15 h at the same temperature, and then treated with water (50 ml). The mixture was extracted with ethyl ether (3×50 ml) and the organic extracts were collected, washed with brine (3×100 ml) and dried. Evaporation of the solvent gave a crude product which was chromatographed (ethyl acetate/n-hexane 9:2 as eluent) to afford pure ST1973, 301 mg, 45%; mp 214-217° C. (toluene); IR: ν 3360 (OH, NH), 2800 (COOH) cm⁻¹; ¹H-NMR (DMSO-d₆): δ 6.63 (d, 2H, Jₒ=8.7 Hz, benzene C3-H and C5-H), 6.92 (d, 1H, J=8.0 Hz, naphthalene H), 7.27 (d, 1H, J=8.0 Hz, naphthalene H), 7.50 (m, 2H, naphthalene H), 7.69 (d, 2H, Jₒ=8.7 Hz, benzene C2-H e C6-H), 7.85 (m, 1H, naphthalene H), 8.20 (m, 1H, naphthalene H), 8.45 (s broad, 1H, NH), 10.20 (s broad, 1H, OH), 12.15 (s broad, 1H, COOH).

### 2-hydroxy-5-[(4-hydroxy-1-naphthyl)amino]benzoic acid (ST3717)

A solution of 2-methoxy-5-(4-methoxy-1-naphthalenylamino)benzoic acid methyl ester ST3245 (500 mg, 1.5 mmol) in dichloromethane (18 ml) was added dropwise to 1M BBr₃ (7.8 ml, 7.8 mmol) in the same solvent at −45° C., under argon atmosphere. The mixture was stirred for 1 h at the same temperature, then warmed at room temperature and stirred for 15 h. After treatment with water (50 ml), the mixture was extracted with ethyl ether (3×50 ml) and the organic extracts were collected, washed with brine (3×100 ml) and dried. Evaporation of the solvent gave a crude product (300 mg), which was chromatographed (ethyl acetate as eluent) to afford pure ST3717 (75 mg, 17%); mp 175° C. dec; IR: ν 3350 (OH, NH), 3000 (COOH) 1635 (CO) cm⁻¹; ¹H-NMR (DMSO-d₆): δ 6.73 (d, 1H, Jₒ=8.7 Hz, benzene C3-H), 6.82 (d, 1H, J=8.0 Hz, naphthalene H), 6.97 (dd, 1H, Jₒ=8.7 Hz, Jₘ=2.7 Hz, benzene C4-H), 7.05 (d, 1H, J=8.0 Hz, naphthalene H), 7.20 (d, 1H, Jₘ=2.7 Hz, benzene C6-H), 7.44-7.49 (m, 2H, naphthalene C6-H and C7-H), 7.99 (m, 1H, naphthalene H), 8.15 (m, 1H, naphthalene H), 9.85 (s broad, 3H, OH, COOH and NH).

### 2-hydroxy-5-[(4-hydroxy-1-naphthyl)amino]benzoic acid hydrochloride (ST2511)

Acetyl chloride (50 mg, 0.6 mmol) was carefully added in methanol (1 ml) cooled at 0° C., under argon stream. Then, a solution of ST3717 (420 mg, 1.8 mmol) in methanol (13 ml) was added dropwise while the hydrochloric solution was gently stirred. After 15 min the solution was concentrated, isopropylic ether (50 ml) was added and the suspension was stirred at 0° C. for 10 min. The precipitate that formed was filtered, washed with cool methanol (1 ml) and then with isopropylic ether (3×2 ml) to give ST2511 (80 mg, 40%). mp 220° C. dec.

### Step iv—Preparation of 4-anilino-1-naphthol (ST2176)

A solution of 4-methoxy-N-phenyl-1-naphthalenamine (ST2173) (600 mg, 2.4 mmol) in dichloromethane (27 ml) was added dropwise to IM BBr₃ (12.6 ml, 12.6 mmol) in the same solvent at −45° C., under argon atmosphere. The mixture was stirred for 15 h at the same temperature, and then treated with water (50 ml). The mixture was extracted with ethyl ether (3×50 ml) and the organic extracts were collected, washed with brine (3×100 ml) and dried. Evaporation of the solvent gave a crude product (630 mg), which was chromatographed (ethyl acetate/n-hexane 1:3 as eluent) to afford pure ST2176 (490 mg, 88%). Oil; IR: ν 3375 (OH, NH) cm⁻¹; ¹H-NMR (CDCl₃): δ 5.30 and 5.65 (2s broad, 2H, OH and NH), 6.75-6.87 (m, 4H, naphthalene H and benzene C2-H and C6-H), 7.15-7.30 (m, 3H, benzene C3-H, C4-H and C5-H), 7.50-7.57 (m, 2H, naphthalene H), 8.05 (m, 1H, naphthalene H), 8.25 (m, 1H, naphthalene H).

The following derivatives were obtained with the same procedure reported above.

### 4-(4-hydroxyanilino)-1-naphthol (ST2177)

The solvents used for the preparation of ST2177 were purged with argon. The compound partially decomposed during chromatography. 15 h; ethyl acetate/n-hexane 1:1; 100%; mp 74° C. dec; IR: ν 3350 (OH, NH) cm⁻¹; ¹H-NMR (DMSO-d₆): δ 6.55-7.00 (m, 7H, naphthalene H, benzene H and NH), 7.38-7.47 (m, 2H, naphthalene H), 7.95-8.13 (m, 2H, naphthalene H), 8.67 and 9.67 (2s broad, 2H, OH).

methyl 4-[(4-hydroxy-1-naphthyl)amino]benzoate (ST2178): 15 h; ethyl acetate/n-hexane 1:2; 58%; mp 188-190° C. (benzene/n-hexane); IR: ν 3370 (NH, OH), 1680 (CO) cm⁻¹; ¹H-NMR (DMSO-d₆): δ 3.73 (s, 3H, CH₃), 6.60-6.67 (m, 2H, benzene C3-H and C5-H), 6.89 (d, 1H, J=8.0 Hz, naphthalene H), 7.24 (d, J=8.0 Hz, naphthalene H), 7.44-7.48 (m, 2H, naphthalene H), 7.66-7.71 (m, 2H, naphthalene H), 7.68 (m, 2H, benzene C2-H and C6-H), 7.82 (m, 1H, naphthalene H), 8.17 (m, 1H, naphthalene H), 8.50 (s broad, 1H, NH), 10.18 (s broad, 1H, OH).

### Step v—Preparation of 4-[(4-methoxy-1-naphthyl)amino]benzoic acid (ST1972)

A solution of ST3244 (500 mg, 1.5 mmol) and 1N NaOH (3.7 ml) in THF/ethanol 1:1 (20 ml) was refluxed for 3.5 h while stirring. Then the mixture was poured onto crushed ice and extracted with ethyl acetate (30 ml). The aqueous layer was treated with 1N HCl until pH 3 and then extracted with ethyl acetate (3×50 ml). The organic extracts were collected, washed with brine (3×100 ml), dried and the solvent was removed to yield ST1972 (240 mg, 50%). Mp 153-154° C. (isopropanol); IR: ν 3400 (NH), 3000 (OH), 1650 (CO) cm⁻¹; ¹H-NMR (DMSO-d₆): δ 4.01 (s, 3H, CH₃), 6.70 (d, 2H, Jₒ=8.5 Hz, benzene C3-H and C5-H), 7.01 (d, 1H, J=8.0 Hz, naphthalene C3-H), 7.39 (d, 1H, J=8.0 Hz, naphthalene C2-H), 7.55 (m, 2H, naphthalene C6-H and C7-H), 7.71 (d, 2H, Jₒ=8.5 Hz, benzene C2-H and C6-H), 7.90 (m, 1H, naphthalene H), 8.20 (m, 1H, naphthalene H), 8.54 (s broad, 1H, NH).

The following derivative were obtained with a similar procedure.

2-methoxy-5-[(4-methoxy-1-naphthyl)amino]benzoic acid (ST2174): 3.5 h; 50%; mp 153-154° C. (isopropanol); IR: ν 3300 (NH), 3160 (OH), 1690 (CO) cm⁻¹; ¹H-NMR (DMSO-d₆): δ 3.76 (s, 3H, OCH₃), 3.97 (s, 3H, OCH₃), 6.91-6.98 (m, 3H, naphthalene H and benzene C3-H and C4-H), 7.16 (m, 1H, benzene C2-H), 7.21 (d, 1H, J=8.2 Hz, naphthalene H), 7.53 (m, 2H, naphthalene H), 7.76 (s broad, 1H, NH), 8.05 and 8.20 (2m, 2H, naphthalene H), 12.50 (s broad, 1H, OH).

### Example 2

### Preparation of Compounds of Formula (I) According to Synthetic Scheme 2

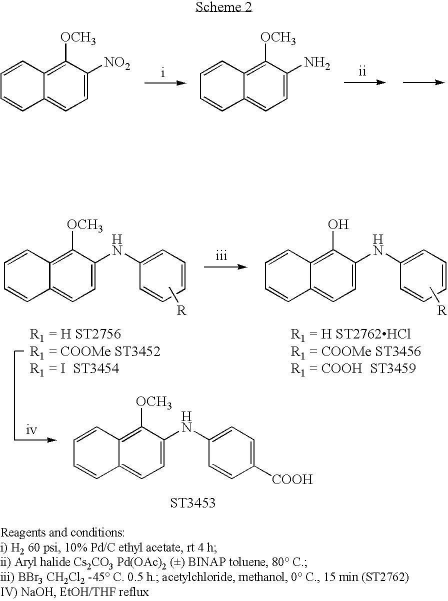

### Step i—Preparation of 1-methoxy-2-naphthalenamine

1-Methoxy-2-naphthalenamine was obtained with the same procedure reported for 4-methoxy-1-naphthalenamine using 1-methoxy-2-nitronaphthalene (3.70 g, 18.0 mmol) as starting material. The 1-methoxy-2-naphthylenamine (3.12 g, 100%) obtained was used for the next reaction without further purification.

### Step ii—Preparation of (1-methoxy-2-naphthyl)phenylamine (ST2756)

A dried flask was purged with argon and charged with (±) BINAP (70 mg, 0.11 mmol) and capped with a rubber septum. The flask was purged with argon and toluene (9.7 ml) was added. The mixture was heated to 80° C. with stirring until the BINAP dissolved (˜1 min). The solution was cooled to room temperature, the septum was removed, and palladium acetate (16 mg, 0.07 mmol) was added. The flask was recapped with the septum and then purged with argon (for 30 sec). The mixture was stirred at room temperature for 1 min, the 1-methoxy-2-naphthalenylamine (600 mg, 3.5 mmol) dissolved in toluene (1.5 ml) and bromobenzene (455 mg, 2.9 mmol) were added, the septum was removed, and cesium carbonate (1.31 g, 4.0 mmol) was added. Additional toluene (7 ml) was added, then the flask was recapped with the septum, and purged with argon again. The mixture was heated to 80° C. with stirring for 16 h. The mixture was cooled to room temperature, diluted with ether, filtered, and concentrated in vacuo. The crude product was then purified by column chromatography (ethyl acetate/n-hexane 1:1 as eluent) to obtain 854 mg (83%) of pure ST2756 mp 43-45° C. (n-hexane); IR: ν 3395 (NH), cm⁻¹; ¹H-NMR (DMSO-d₆): δ 3.80 (s, 3H, CH₃), 6.85 (m, 1H, benzene H), 7.07-7.65 (m, 8H, naphthalene H and benzene H), 7.84 (m, 1H, naphthalene H), 7.93 (m, 1H, naphthalene H), 7.99 (m, 1H, naphthalene H).

### Methyl-4-[(1-methoxy-2-naphthyl)amino]benzoate (ST3452)

A dried flask was purged with argon and charged with (±) BINAP (210 mg, 0.34 mmol) and capped with a rubber septum. The flask was purged with argon and toluene (31 ml) was added. The mixture was heated to 80° C. with stirring until the BINAP dissolved. The solution was cooled to room temperature, the septum was removed, and palladium acetate (50 mg, 0.23 mmol) was added. The flask was recapped with the septum and then purged with argon. The mixture was stirred at room temperature for 1 min, the 1-methoxy-2-naphthalenamine (1.93 g, 11.16 mmol) (see Scheme 2 Step i), dissolved in toluene (6 ml) and methyl 4-bromobenzoate (2.00 g, 13.02 mmol), the septum was removed, and cesium carbonate (4.24 g, 13.02 mmol) was added. Additional toluene (23 ml) was added, then the flask was recapped with the septum, and purged with argon again. The mixture was heated to 80° C. under stirring for 4 h and 10 min. The mixture was cooled to room temperature, diluted with ether, filtered, and concentrated in vacuo. The crude product (4.06 g) was then purified by column chromatography (Chloroform/ethyl acetate 9:1 as eluent) to obtain 2.78 g (97%) of pure ST3452. p.f. 153-154° C. (ligroina); IR: ν 3327 (NH), 1691 (CO) cm⁻¹; ¹H-NMR (CDCl₃): δ 3.95 (s, 3H, CH₃), 7.14 (d, 2H, Jₒ=8.8 Hz, benzene C2-H and C6-H), 7.44-7.48 (m, 1H, naphthalene H), 7.55-7.59 (m, 1H, naphthalene H), 7.64-7.69 (m, 2H, naphthalene H), 8.03 (d, 2H, Jₒ=8.8 Hz, benzene C3-H and C5-H), 8.10 (m, 1H, naphthalene H).

### N-(4-iodophenyl)-1-methoxynaphthalen-2-amine(ST3454)

A dried flask was purged with argon and charged with (±) BINAP (125 mg, 0.20 mmol) and capped with a rubber septum. The flask was purged with argon and toluene (19 ml) was added. The mixture was heated to 80° C. with stirring until the BINAP dissolved. The solution was cooled to room temperature, the septum was removed, and palladium acetate (30 mg, 0.135 mmol) was added. The flask was recapped with the septum and then purged with argon. The mixture was stirred at room temperature for 1 min, 1-methoxy-2-naphthalenamine (1.12 g, 6.5 mmol) (see Scheme 2 Step i), dissolved in toluene (4 ml) and 1,4-diiodobenzene (1.78 g, 5.4 mmol), the septum was removed, and cesium carbonate (2.46 g, 7.56 mmol) was added. Additional toluene (15 ml) was added, then the flask was recapped with the septum, and purged with argon again. The mixture was heated to 80° C. under stirring for 19 h. The mixture was cooled to room temperature, diluted with ether, filtered, and concentrated in vacuo. The crude product (3.72 g) was purified by column chromatography (Chloroform/petroleum ether 1:1 as eluent) to obtain 740 mg (37%) of pure ST3454; p.f. 83-84° C. (n-hexane); IR: v 3327 (NH) cm⁻¹; ¹H-NMR (CDCl₃): δ 3.96 (s, 3H, CH₃), 6.97 (d, 2H, Jₒ=8.8 Hz, benzene C2-H and C6-H), 7.40-7.43 (m, 1H, naphthalene H), 7.55 (d, 2H, Jₒ=8.8 Hz, benzene C3-H and C5-H), 7.57 (m, 1H, naphthalene H), 7.85 and 7.07 (2m, 2H, naphthalene H).

### Step iii—Preparation of 1-hydroxy-N-phenylnaphthalen-2-aminium chloride (ST2762)

A solution of 1-Methoxy-N-phenyl-2-naphthalenamine (ST2756) (705 mg, 2.4 mmol) in dichloromethane (27 ml) was added dropwise to 1M BBr₃ (12.6 ml, 12.6 mmol) in the same solvent at −45° C., under argon atmosphere. The mixture was stirred for 30 minutes at the same temperature, and then treated with water (50 ml). The mixture was extracted with ethyl ether (3×50 ml) and the organic extracts were collected, washed with brine (3×100 ml) and dried. Evaporation of the solvent gave a crude product (630 mg), which was chromatographed (ethyl acetate/n-hexane 1:3 as eluent) to afford pure 571 mg, 88%; acetyl chloride (150 mg, 1.9 mmol) was carefully added in methanol (3 ml) cooled at 0° C., under argon stream. Then, a solution of product pure (420 mg, 1.8 mmol) in methanol (3 ml) was added dropwise while the hydrochloride solution was gently stirred. After 15 min the solution was concentrated, isopropylic ether (17 ml) was added and the suspension was stirred at 0° C. for 10 min. The precipitate that formed was filtered, washed with cool methanol (1 ml) and then with isopropylic ether (3×2 ml) to give (170 mg, 33.5%) di ST2762. Mp>300° C.; IR: ν 3150 (NH e OH) cm⁻¹; ¹H-NMR (DMSO-d₆): δ 6.70 (m, 1H, benzene H), 6.81-6.88 (m, 2H, benzene H), 7.07-7.16 (m, 2H, naphthalene H), 7.31-7.42 (m, 4H, benzene H and naphthalene H), 7.77 (m, 1H, naphthalene H), 8.11 (m, 1H, naphthalene H).

### Methyl 4-[(1-hydroxy-2-naphthyl)amino]benzoate (ST3456)

### 4-[1-hydroxy-2-naphthyl)amino]benzoic acid (ST3459)

A solution of ST3452 (1.46 g, 4.75 mmol) in dichloromethane (54 ml) was added dropwise to 1M BBr₃ Dichlorometane solution (23.7 ml, 23.7 mmol) at −45° C., under argon atmosphere. The mixture was stirred for 19 h and 40 min at the same temperature and also 35 min at room temperature. The mixture was diluted with water (100 ml) and extracted with ethyl acetate (3×100 ml); the organic layers were collected, washed with brine (3×100 ml), dried and concentrated under vacuo obtaining a crude product (1.02 g), which was purified by column chromatography (ethyl acetate/n-hexane 1:1 as eluent) to afford ST3456 (610 mg) with same impurities and pure ST3459 (460 mg). ST3459: p.f. 210 (dec) ° C. (MeOH); IR: ν 3426 (OH, COOH), 3353 (NH), 1654 (CO) cm⁻¹; ¹H-NMR (DMSO-d₆): δ 6.78-6.80 (m, 2H, benzene C2-H e C6-H), 7.33-7.36 (m, 1H, naphthalene H), 7.42-7.50 (m, 3H, naphthalene H), 7.74-7.77 (m, 2H, benzene C3-H and C5-H), 7.84 7.86 (m, 1H, naphthalene H), 8.18 (s broad, 1H, NH), 8.19-8.21 (m, 1H, naphthalene H), 9.40 (s broad, 1H, OH), 12.20 (s broad, 1H, COOH).

Unclear ST3456 was purified by column chromatography (acetone/n-hexane 1:4 as eluent) obtaining pure ST3456 (500 mg). p.f. 175-176° C. (toluene); IR: ν 3334 (OH and NH), 1684 (CO) cm⁻¹; ¹H-NMR (DMSO-d₆): δ 3.79 (s, 3H, CH₃), 6.79 (d, 2H, benzene H), 6.64 (m, 1H, benzene H), 7.17 (m, 1H, naphthalene H), 7.28-7.31 (m, 2H, naphthalene H), 7.39 (m, 1H, Jₒ=8.8 Hz, benzene C2-H and C6-H), 7.35 (m, 1H, naphthalene H), 7.45-7.51 (m, 3H, naphthalene H), 7.77 (m, 1H, Jₒ=8.8 Hz, benzene C3-H and C5-H), 7.85 and 8.21 (2m, 2H, naphthalene H), 8.25 (s broad, 1H, NH), 9.40 (s broad, 1H, OH).

### Step iv—Preparation of

### 4-[(1-methoxy-2-naphthyl)amino]benzoic acid (ST3453)

A solution of ST3452 (700 mg, 2.3 mmol) and 1N NaOH (5.75 ml) in THF/ethanol 1:1 was refluxed for 1 h and 40 min under stirring. Then the mixture was poured onto crushed ice and extracted with ethyl acetate (1×20 ml). The aqueous layer was treated with 1N HCl and then extracted with ethyl acetate (3×100 ml). The organic extracts were collected, washed with brine (3×100 ml), dried and the solvent was removed to yield ST3453 (700 mg, 100%); p.f. 233-235° C. (EtOH); IR: ν 3403 (COOH e NH), 1691 (CO) cm⁻¹; ¹H-NMR (DMSO-d₆): δ 3.80 (s, 3H, CH₃), 7.01 (d, 2H, Jₒ=8.6 Hz, benzene C2-H and C6-H), 7.46-7.59 (m, 1H, naphthalene H), 7.72 (m, 1H, naphthalene H), 7.82 (d, 2H, Jₒ=8.6 Hz, benzene C3-H and C5-H 7.64-7.69), 7.93 and 8.08 (2m, 2H, naphthalene H), 8.59 (s broad, 1H, NH), 12.32 (s broad, 1H, COOH).

### Example 3

### Preparation of Compounds of Formula (I) According to Synthetic Scheme 3

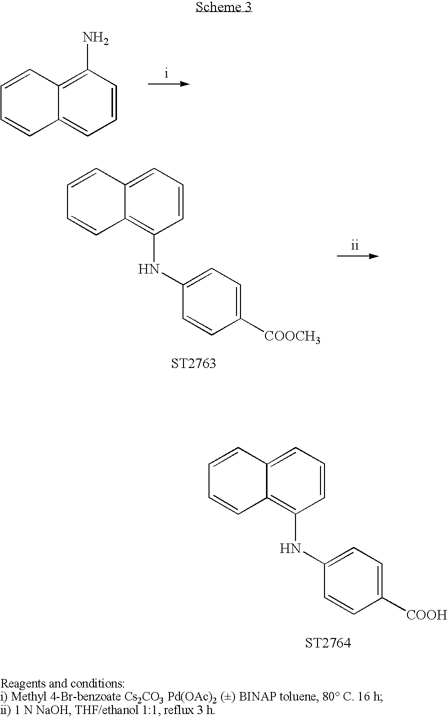

### Step i—Preparation of methyl 4-(1-naphthylamino)benzoate (ST2763)

A dried flask was purged with argon and charged with (±) BINAP (70 mg, 0.11 mmol) and capped with a rubber septum. The flask was purged with argon and toluene (9.7 ml) was added. The mixture was heated to 80° C. with stirring until the BINAP dissolved (˜1 min). The solution was cooled to room temperature, the septum was removed, and palladium acetate (16 mg, 0.07 mmol) was added. The flask was recapped with the septum and then purged with argon (for ˜30 sec). The mixture was stirred at room temperature for 1 min, the 1-naphthalenylamine (600 mg, 3.5 mmol) dissolved in toluene (1.5 ml) and methyl-4-bromobenzoate (623 mg, 2.9 mmol) were added, the septum was removed, and cesium carbonate (1.31 g, 4.0 mmol) was added. Additional toluene (7 ml) was added, then the flask was recapped with the septum, and purged with argon again. The mixture was heated to 80° C. with stirring for 16 h. The mixture was cooled to room temperature, diluted with ether, filtered, and concentrated in vacuo. The crude product was then purified by column chromatography (chloroform/petroleum ether 3:1 as eluent) to obtain 771 mg (96%) of pure ST2763. mp 130-132° C. (toluene); IR: ν 3340 (NH), 1694 (CO) cm⁻¹; ¹H-NMR (DMSO-d₆): δ 3.79 (s, 3H, CH₃), 6.95 (m, 2H, benzene C3-H and C5-H), 7.45-7.60 (m, 4H, naphthalene H), 7.73 (m, 1H, naphthalene H), 7.79 (m, 2H, benzene C2-H and C6-H), 7.98 (m, 1H, naphthalene H), 8.06 (m, 1H, naphthalene H), 8.88 (s broad, 1H, NH).

### Step ii—4-(1-naphthylamino)benzoic acid (ST2764)

A solution of ST2763 (415 mg, 1.5 mmol) and 1N NaOH (3.7 ml) in THF/ethanol 1:1 (20 ml) was refluxed for 3 h while stirring. Then the mixture was poured onto crushed ice and extracted with ethyl acetate (30 ml). The aqueous layer was treated with 1N HCl until pH 3 and then extracted with ethyl acetate (3×50 ml). The organic extracts were collected, washed with brine (3×100 ml), dried and the solvent was removed to yield ST2764 232 mg, (59%). mp 227-229° C. (toluene); IR: ν 3390 (NH), 2900 (OH), 1670 (CO) cm⁻¹; ¹H-NMR (DMSO-d₆): δ 6.95 (m, 2H, benzene C3-H and C5-H), 7.46-7.60 (m, 4H, naphthalene H), 7.72 (m, 1H, naphthalene H), 7.78 (m, 2H, benzene C2-H and C6-H), 7.96 (m, 1H, naphthalene H), 8.07 (m, 1H, naphthalene H), 8.81 (s broad, 1H, NH), 12.29 (s broad, 1H, OH).

### Example 4

### Preparation of Compounds of Formula (I) According to Synthetic Scheme 4

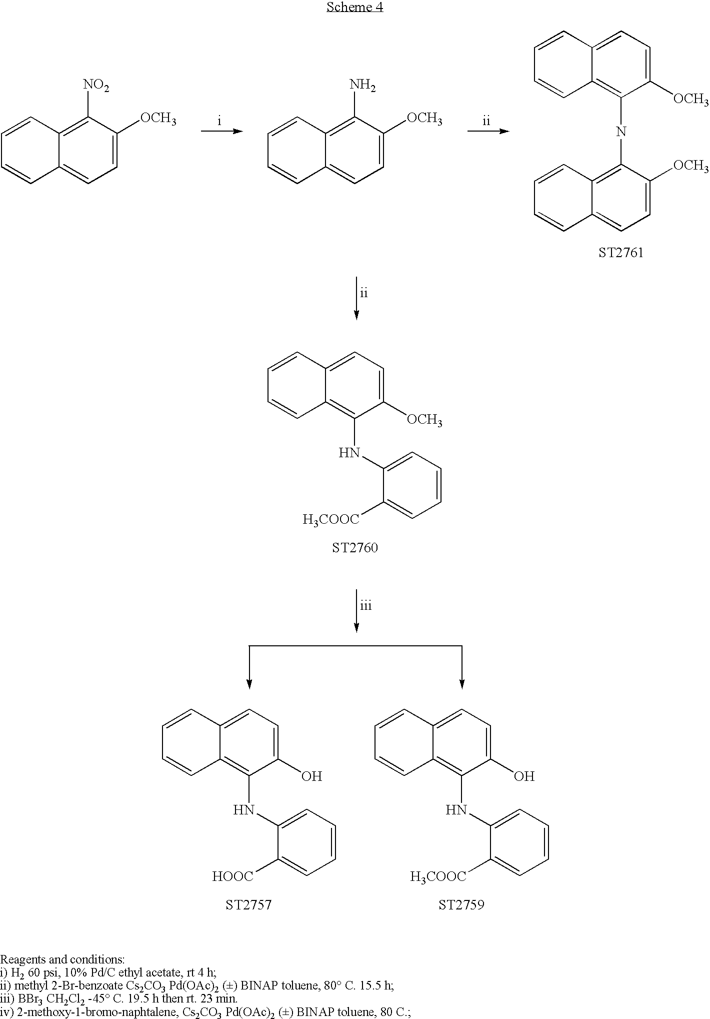

### Step i—Preparation of 2-methoxy-1-naphthalenamine

2-Methoxy-1-naphthalenamine was obtained with the same procedure reported above, (step i, scheme 1) using 2-methoxy-1-nitronaphthalene (3.00 g, 14.8 mmol) as starting material. The 2-methoxy-1-naphthylenamine (2.6 g, 100%) obtained was used for the next reaction without further purification.

### Step ii—Preparation of methyl-2-(2-methoxy-1-naphthalenylamino)benzoate (ST2760)

A dried flask was purged with argon and charged with (±) BINAP (70 mg, 0.11 mmol) and capped with a rubber septum. The flask was purged with argon and toluene (9.7 ml) was added. The mixture was heated to 80° C. with stirring until the BINAP dissolved (˜1 min). The solution was cooled to room temperature, the septum was removed, and palladium acetate (16 mg, 0.07 mmol) was added. The flask was recapped with the septum and then purged with argon (for 30 sec). The mixture was stirred at room temperature for 1 min, the 2-methoxy-1-naphthalenylamine (606 mg, 3.5 mmol) dissolved in toluene (1.5 ml) and methyl-2-bromobenzoate (615 mg, 2.9 mmol) were added, the septum was removed, and cesium carbonate (1.31 g, 4.0 mmol) was added. Additional toluene (7 ml) was added, then the flask was recapped with the septum, and purged with argon again. The mixture was heated to 80° C. with stirring for 15.5 h. The mixture was cooled to room temperature, diluted with ether, filtered, and concentrated in vacuo; the crude product was purified by column chromatography ethyl acetate/n-hexane 1:5, to obtain ST2760 890 mg 100%; mp 144-146° C. (cyclohexane); IR: ν 3321 (NH), 1681 (CO) cm⁻¹; ¹H-NMR (DMSO-d₆): δ 3.86 (s, 3H, CH₃), 3.89 (s, 3H, CH₃), 6.09 (m, 1H, benzene H), 6.66 (m, 1H, benzene H), 7.18 (m, 1H, naphthalene H), 7.35-7.45 (m, 2H, benzene H and naphthalene H), 7.57 (m, 1H, naphthalene H), 7.68 (m, 1H, benzene H), 7.88-7.95 (m, 3H, naphthalene H), 9.17 (s broad, 1H, NH).

### Step iii—Preparation of methyl 2-[(2-hydroxy-1-naphthyl)amino]benzoate (ST2759) and 2-[(2-hydroxy-1-naphthyl)amino]benzoic acid (ST2757)

A solution of ST2760 (736 mg, 2.4 mmol) in dichloromethane (27 ml) was added dropwise to 1M BBr₃ (12.6 ml, 12.6 mmol) in the same solvent at −45° C., under argon atmosphere. The mixture was stirred for 19.5 h at the same temperature, and then warmed at room temperature and stirred for 23 min; then treated with water (50 ml). The mixture was extracted with ethyl ether (3×50 ml) and the organic extracts were collected, washed with brine (3×100 ml) and dried. Evaporation of the solvent gave a crude product, which was chromatographed (ethyl acetate/n-hexane 1:2 as eluent); first eluates ST2759, 316 mg, 45%; mp 157-158° C. (cyclohexane); IR: ν 3407 (OH), 3319 (NH), 1681 (CO) cm⁻¹; 19.5 h; ¹H-NMR (DMSO-d₆): δ 3.88 (s, 3H, CH₃), 6.10 (m, 1H, benzene H), 6.64 (m, 1H, benzene H), 7.17 (m, 1H, naphthalene H), 7.28-7.31 (m, 2H, naphthalene H), 7.39 (m, 1H, benzene H), 7.62 (m, 1H, benzene H), 7.77 (m, 1H, naphthalene H), 7.84-7.95 (m, 2H, naphthalene H), 9.05 (s broad, 1H, NH), 9.78 (s broad, 1H, OH). Further elution afforded ST2757,368 mg 55%; mp 215-216° C. (toluene); IR: ν 3361 (OH, COOH), 3325 (NH), 1659 (CO) cm⁻¹; ¹H-NMR (DMSO-d₆): δ 6.09 (m, 1H, benzene H), 6.62 (m, 1H, benzene H), 7.14 (m, 1H, naphthalene H), 7.28-7.31 (m, 2H, naphthalene H), 7.39 (m, 1H, benzene H), 7.62 (m, 1H, benzene H), 7.75 (m, 1H, naphthalene H), 7.83-7.89 (m, 2H, naphthalene H), 9.28 (s broad, 1H, NH), 9.76 (s broad, 1H, OH), 12.86 (s broad, 1H, COOH).

### Step iv—Preparation of 2-methoxy-N-(2-methoxy-1-naphthyl)naphthalen-1-amine (ST2761)

A dried flask was purged with argon and charged with (±) BINAP (200 mg, 0.323 mmol) and capped with a rubber septum. The flask was purged with argon and toluene (29 ml) was added. The mixture was heated to 80° C. with stirring until the BINAP dissolved (˜1 min). The solution was cooled to room temperature, the septum was removed, and palladium acetate (50 mg, 0.218 mmol) was added. The flask was recapped with the septum and then purged with argon. The mixture was stirred at room temperature for 1 min, the 2-methoxynaphthalen-1-yl-amine (1.81 g, 10.5 mmol) dissolved in toluene (4.5 ml) and 2-methoxy-1-bromonaphthalene (2.07 g, 8.73 mmol) were added, the septum was removed, and cesium carbonate (3.98 g, 12.2 mmol) was added. Additional toluene (21.2 ml) was added, then the flask was recapped with the septum, and purged with argon again. The mixture was heated to 80° C. with stirring for 20 h. The (±) BINAP (200 mg, 0.323 mmol), palladium acetate (50 mg, 0.218 mmol) and toluene (29 ml) were added. The mixture was heated to 80° C. with stirring for 15 h. The (±) BINAP (200 mg, 0.323 mmol), palladium acetate (50 mg, 0.218 mmol) and toluene (29 ml) were added. The mixture was heated to 80° C. with stirring for 24 h. The (±) BINAP (200 mg, 0.323 mmol), palladium acetate (50 mg, 0.218 mmol) and toluene (29 ml) were added The mixture was heated to 80° C. with stirring for 20 h. The mixture was cooled at room temperature, diluted with ether, filtered, and concentrated in vacuo. The crude product (5.03 g) was then purified by column chromatography (ethyl acetate/n-hexane 1:5 as eluent) to obtain 1.69 g (59%) of pure ST2761 (Oil). IR: ν 3380 (NH), cm⁻¹; ¹H-NMR (DMSO-d₆): δ 3.55 (s, 6H, CH₃), 7.05 (s broad, 1H, NH), 7.20-7.38 (m, 6H, naphthalene H), 7.58 (m, 2H, naphthalene H), 7.81-7.93 (m, 4H, naphthalene H).

### Example 5

### Preparation of Compounds of Formula (I) According to Synthetic

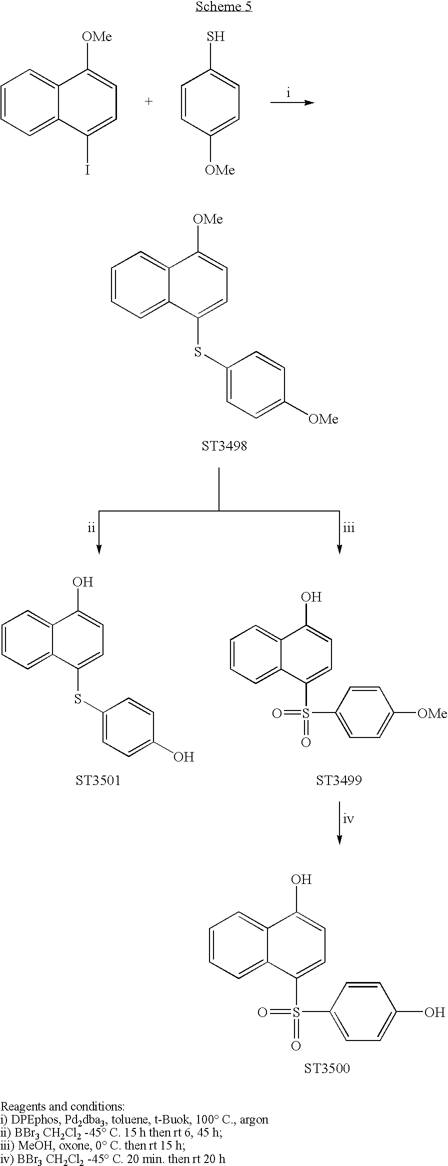

### Step i—Preparation of

### 1-methoxy-4-[(4-methoxyphenyl)thio]naphthalene(ST3498)

A dried flask was charged with Pd₂ dba₃ (130 mg, 0.141 mmol) dissolved in degassed toluene (115 ml), treated with DPEphos (150 mg, 0.282 mmol) and purged with argon. The mixture was stirred at room temperature for 3 min, then 1-methoxy-4-iodonaphthalene (4 g, 14.1 mmol) and 4-methoxythiophenol (2.02 g, 14.1 mmol, 1.77 ml) were added under argon atmosphere. t-BuOK (1.74 g, 15.5 mmol) was added and the flask purged with argon. The mixture was stirred for 2 h at 100° C., cooled at room temperature, filtered on celite cake and the filtered was concentrated under vacuo. The crude product (6.19 g) was purified by column chromatography (n-hexane/acetone 10:1 as eluent) obtaining the unclear final product (3.57 g), which was further purified by crystallization (n-hexane) obtaining 2.70 g (65%) of pure ST3498. p.f. 83-85° C. (n-hexane); IR: v 2937 (CH) cm⁻¹; ¹H-NMR (acetone-d₆): δ 3.78 (s, 3H, CH₃), 4.10 (s, 3H, CH₃), 6.88 (d, 2H, Jₒ=8.86 Hz, benzene H), 7.03 (d, 1H, Jₒ=8.01 Hz, naphthalene C2-H), 7.20 (d, 2H, Jₒ=8.86 Hz, benzene H), 7.57-7.64 (m, 2H, naphthalene C6-H and C7-H), 7.75 (d, 1H, Jₒ=8.01 Hz, naphthalene C3-H), 8.34 and 8.40 (2m, 2H, naphthalene C5-H and C8-H).

### Step iii—Preparation of

### 1-methoxy-4-[(4-methoxyphenyl)sulfonyl]naphthalene (ST3499)

ST3498 (1 g, 3.4 mmol) was dissolved in MeOH (84 ml). At 0° C. a solution of Oxone® (6.27 g, 10.2 mmol) in water (20 ml) was added. The mixture was stirred for 16 h at room temperature. The mixture was poured in water, extracted with ethyl acetate (3×100 ml), the collected organic layers washed with brine (3×100 ml) and dried over Na₂SO₄ anhydrous. The solvent was evaporated in vacuo obtaining a crude product which was purified by column chromatography (chloroform as eluent) affording the pure product ST3499 (82%); p.f. 165-167° C. (toluene). IR: v 2900 (CH) cm⁻¹; ¹H-NMR (DMSO-d₆): δ 3.82 (s, 3H, CH₃), 4.12 (s, 3H, CH₃), 7.11 (d, 2H, Jₒ=8.69 Hz, benzene H), 7.25 (d, 1H, Jₒ=8.47 Hz, naphthalene C2-H), 7.64 and 7.72 (2m, 2H, naphthalene C6-H and C7-H), 7.90 (d, 2H, Jₒ=8.69 Hz, benzene H), 8.30 (d, 1H, Jₒ=8.47 Hz, naphthalene C3-H), 8.46 and 8.52 (2m, 2H, naphthalene C5-H and C8-H).

### Step iv—Preparation of

### 4-[(4-hydroxyphenyl)sulfonyl]-1-naphthol (ST3500)

A solution of 1-methoxy-4-[(4-methoxyphenyl)sulphonyl]-naphthalene (ST3499) (1 g, 3 mmol) in dichloromethane (35 ml) was added dropwise to 1M BBr₃ dichlorometane solution (15.9 ml, 15.9 mmol) at −45° C., under argon atmosphere. The mixture was stirred for 20 min at the same temperature and 20 h at room temperature. The mixture was diluted with water (100 ml) and extracted with ethyl acetate (3×100 ml); the organic layers were collected, washed with brine (3×100 ml), dried and evaporated under vacuo obtaining a crude product (900 mg), which was purified by column chromatography (ethyl acetate/chloroform 1:2 as eluent) to afford 520 mg of pure ST3500 (58%); p.f. 203-205° C. (toluene). IR: v 3300 (OH) cm⁻¹; ¹H-NMR (DMSO-d₆): δ 6.90 (d, 2H, Jₒ=8.77 Hz, benzene H), 7.07 (d, 1H, Jₒ=8.29 Hz, naphthalene C2-H), 7.58 and 7.67 (2m, 2H, naphthalene C6-H and C7-H), 7.77 (d, 2H, Jₒ=8.77 Hz, benzene H), 8.28-8.31 (m, 2H, naphthalene C5-H and C8-H), 8.48 (d, 1H, Jₒ=8.29 Hz, naphthalene C3-H), 10.54 and 11.51 (2s broad, 2H, OH).

### Step ii—Preparation of

### 4-[(4-hydroxyphenyl)thio]-1-naphthol (ST3501)

A solution of 1-methoxy-4-[(4-methoxyphenyl)thio]-naphthalene ST3498 (800 mg, 2.7 mmol) in dichloromethane (33 ml) was added dropwise to 1M BBr₃ Dichlorometane solution (14.1 ml, 14.1 mmol) at −45° C., under argon atmosphere. The mixture was stirred for 15 h and 35 min at the same temperature and 6 h and 45 min at room temperature. The mixture was diluted with water (100 ml) and extracted with ethyl acetate (3×100 ml); the organic layers were collected, washed with brine (3×100 ml), dried and evaporated under vacuo obtaining a crude product, which was purified by column chromatography (ethyl acetate/n-hexane 2:5 as eluent) to afford 440 mg of pure ST3501 (61%); p.f. 161-163° C. (toluene). IR: v 3255 (OH) cm⁻¹; ¹H-NMR (DMSO-d₆): δ 6.70 (d, 2H, Jₒ=8.69 Hz, benzene H), 6.93 (d, 1H, Jₒ=7.89 Hz, naphthalene C2-H), 7.06 (d, 2H, Jₒ=8.69 Hz, benzene H), 7.51-7.62 (m, 3H, C3-H, naphthalene C6-H and C7-H), 8.23 and 8.27 (2m, 2H, naphthalene C5-H and C8-H), 9.52 and 10.61 (2s broad, 2H, OH).

### Example 6

### Preparation of Compounds of Formula (I) According to Synthetic Scheme 6

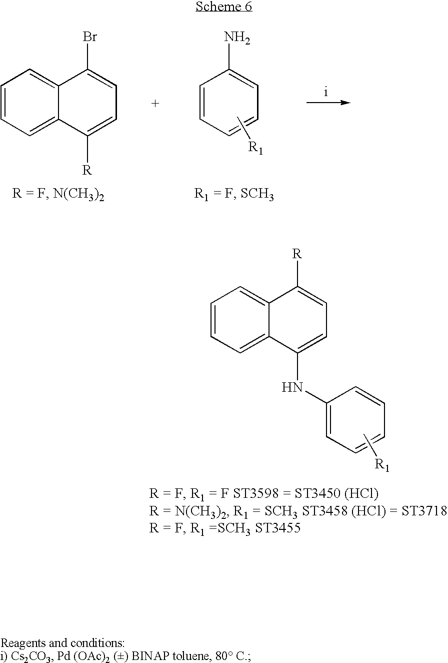

### Step i—Preparation of

### 4-fluoro-N-(4-fluorophenyl)naphthalen-1-amine (ST3598)

A dried flask was purged with argon and charged with (±) BINAP (160 mg, 0.25 mmol) and capped with a rubber septum. The flask was purged with argon and toluene (24 ml) was added. The mixture was heated to 80° C. with stirring until the BINAP dissolved. The solution was cooled to room temperature, the septum was removed, and palladium acetate (40 mg, 0.17 mmol) was added. The flask was recapped with the septum and then purged with argon. The mixture was stirred at room temperature for 1 min, the 4-fluoroaniline (890 mg, 8.04 mmol) dissolved in toluene (3 ml) and 1-bromo-4-fluoronaphthalene (1.50 g, 6.7 mmol) were added, the septum was removed, and cesium carbonate (3.06 g, 9.38 mmol) was added. Additional toluene (18 ml) was added, then the flask was recapped with the septum, and purged with argon again. The mixture was heated to 80° C. under stirring for 5 h and 45 min. The mixture was cooled to room temperature, diluted with ether, filtered, and concentrated in vacuo. The crude product (2.31 g) was then purified by column chromatography (Chloroform as eluent) to obtain 1.87 g (91%) of pure ST3598. p.f. 62-64° C. (not crystallized); IR: v 3395 cm⁻¹ (NH); ¹H-NMR (CDCl₃): δ 5.55 (s broad, 1H, NH), 6.86-6.90 (m, 2H, benzene H), 6.98-7.03 (m, 2H, benzene H), 7.11-7.17 (m, 1H, naphthalene C2-H), 7.24 (m, 1H, naphthalene C3-H), 7.57-7.66 (m, 2H, naphthalene C6-H and C7-H), 8.05 and 8.20 (2m, 2H, naphthalene C5-H and C8-H).

### 4-fluoro-N-(4-fluorophenyl)naphthalen-1-amine hydrochloride (ST3450)

Acetyl chloride (310 mg, 3.9 mmol) was carefully added in methanol (17 ml) cooled at 0° C., under argon stream. A solution of ST3598 (1.00 g, 3.9 mmol) in methanol (1 ml) was added dropwise to the hydrochloric solution gently stirred. After 15 min under stirring at the same temperature, the solution was concentrated and cooled at −18° C. for 5 days to give ST3450 (100%); p.f. 63-65° C.; IR: v 3390 (NH) cm⁻¹; ¹H-NMR (CDCl₃): δ 5.55 (s broad, 1H, NH), 6.88-6.92 (m, 2H, benzene H), 6.99-7.03 (m, 2H, benzene H), 7.11-7.16 (m, 1H, naphthalene C2-H), 7.23-7.26 (m, 1H, naphthalene C3-H), 7.58-7.66 (m, 2H, naphthalene C6-H and C7-H), 8.06 and 8.20 (2m, 2H, naphthalene C5-H and C8-H).

### N,N-dimethyl-N′-[4-(methylthio)phenyl]naphthalene (ST3718)

A dried flask was purged with argon and charged with (±) BINAP (140 mg, 0.22 mmol) and capped with a rubber septum. The flask was purged with argon and toluene (21 ml) was added. The mixture was heated to 80° C. with stirring until the BINAP dissolved. The solution was cooled to room temperature, the septum was removed, and palladium acetate (33 mg, 0.147 mmol) was added. The flask was recapped with the septum and then purged with argon. The mixture was stirred at room temperature for 1 min, the 4-(methylthio)aniline (990 mg, 7.08 mmol) dissolved in toluene (1 ml) and 1-bromo-4-(dimethylamino)naphthalene (1.47 g, 5.9 mmol) dissolved in toluene (1 ml) were added, the septum was removed, and cesium carbonate (2.69 g, 8.26 mmol) was added. Additional toluene (16 ml) was added, then the flask was recapped with the septum, and purged with argon again. The mixture was heated to 80° C. under stirring for 16 h. A solution of (±) BINAP (140 mg, 0.22 mmol) and palladium acetate (33 mg, 0.147 mmol) dissolved in toluene (21 ml) was added and the mixture was stirred at 80° C. for 4 h and 50 min. The mixture was cooled to room temperature, diluted with ether, filtered, and concentrated in vacuo. The crude product (2.87 g) was then purified by column chromatography (Chloroform as eluent) to obtain 1.48 g (81%) of pure ST3718. Oil; IR: v 3381 (NH) cm⁻¹; ¹H-NMR (DMSO-d₆): δ 2.42 (s, 3H, SCH₃), 2.84 (s, 6H, NCH₃), 6.86 (d, 2H, Jₒ=8.8 Hz, benzene C2-H and C6-H), 7.14-7.20 (m, 3H, naphthalene H and benzene C3-H and C5-H), 7-7.56 (m, 2H, naphthalene C2-H and C3-H), 8.07-8.09 (m, 2H, NH and naphthalene H), 8.23 (m, 1H, naphthalene H).

### N,N-dimethyl-N′-[4-(methylthio)phenyl]naphthalene-1,4-diamine dihydrochloridedichloridrate (ST3458)

Acetyl chloride (410 mg, 5.2 mmol) was carefully added in methanol (1 ml) cooled at 0° C., under argon stream. A solution of ST3718 (800 mg, 2.6 mmol) in methanol (4 ml) was added dropwise to the hydrochloric solution gently stirred. The solution was stirred for 15 min at 0° C., diluted with ethyl ether and further stirred for 10 min at 0° C. The precipitate was filtered obtaining ST3458 (88%); p.f. 204-205° C. (isopropyl alcohol); IR: v 3278 (NH) cm⁻¹; ¹H-NMR (DMSO-d₆): δ 2.46 (s, 3H, SCH₃), 3.19 (s, 6H, NCH₃), 4.13 (s broad, 2H, NH), 7.07-7.09 (d, 2H, Jₒ=8.8 Hz, benzene C2-H and C6-H), 7.25-7.27 (m, 3H, naphthalene C3-H and benzene C3-H and C5-H), 7.62-7.73 (m, 3H, naphthalene C2-H, C7-H and C8-H), 8.31-8.45 (m, 3H, naphthalene C5-H, C8-H and NH).

### 4-fluoro-N-[4-(methylthio)phenyl]naphthalen-1-amine (ST3455)

A dried flask was purged with argon and charged with (±) BINAP (160 mg, 0.25 mmol) and capped with a rubber septum. The flask was purged with argon and toluene (24 ml) was added. The mixture was heated to 80° C. with stirring until the BINAP dissolved. The solution was cooled to room temperature, the septum was removed, and palladium acetate (40 mg, 0.17 mmol) was added. The flask was recapped with the septum and then purged with argon. The mixture was stirred at room temperature for 1 min, the 4-(methylthio)aniline (1.12 g, 8.04 mmol) dissolved in toluene (3 ml) and 1-bromo-4-fluoronaphthalene (1.50 g, 6.7 mmol) were added, the septum was removed, and cesium carbonate (3.06 g, 9.38 mmol) was added. Additional toluene (18 ml) was added, then the flask was recapped with the septum, and purged with argon again. The mixture was heated to 80° C. under stirring for 17 h. The mixture was cooled to room temperature, diluted with ether, filtered, and concentrated in vacuo. The crude product (2.85 g) was then purified by column chromatography (chloroform/petroleum ether 1:1 as eluent) to obtain 1.79 g (94%) of pure ST3455; p.f. 71-72° C. (n-hexane); IR: v 3337 (NH) cm⁻¹; ¹H-NMR (DMSO-d₆): δ 2.44 (s, 3H, CH₃), 6.93 (d, 2H, Jₒ=8.7 Hz, benzene C3-H and C5-H), 7.20-7.33 (m, 4H, benzene C2-H and C3-H, naphthalene C2-H and C3-H), 7.63-7.71 (m, 2H, naphthalene C6-H and C7-H), 8.07 and 8.18 (2m, 2H, naphthalene C5-H and C8-H), 8.21 (s broad, 1H, NH).

### Example 7

### General Analytical Methods

Melting points were determined on a Bibby Stuart Scientific SMP1 melting point apparatus and are uncorrected.

Infrared (IR) spectra (Nujol mulls) were recorded on a Perkin-Elmer Spectrum-one spectrophotometer.

¹H NMR spectra were recorded at 400 MHz on a Bruker AC 400 Ultrashield spectrophotometer (400 MHz). Dimethylsulfoxide-d6 99.9% (code 44, 139-2) and deuterochloroform 98.8% (code 41, 675-4) of isotopic purity (Aldrich) were used.

The solvent Column chromatographies were performed on silica gel (Merck; 70-230 mesh) column. All compounds were routinely checked by TLC by using aluminium-baked silica gel plates (Fluka DC-Alufolien Kieselgel 60 F₂₅₄). Developed plates were visualized by UV light. Solvents were reagent grade and, when necessary, were purified and dried by standard methods. Concentration of solutions after reactions and extractions involved the use of rotary evaporator (Büchi) operating at a reduced pressure (ca. 20 Torr). Organic solutions were dried over anhydrous sodium sulfate (Merck).

### Example 8

### Evaluation of anti-aggregating activity of the compounds of formula (I) on the peptide βAmloid₁₋₄₂

The anti-aggregating activity of the compound of formula (I) on the peptide βA₁₋₄₂ is carried out via the binding of the thioflavin T according to the following procedure.

### Preparation of the Non Aggregate β-A₍₁₋₄₂₎

The β-A₍₁₋₄₂₎ was dissolved in a mixture of Acetonitrile and distilled water (CH₃CN/H₂O 1:1) to the final concentration of 1 mg/mL. The solution was divided in aliquots of 2 mL and stored at −80° C. until the use. The work solution was prepared diluting the stock solution five times with H₂O (final concentration 44 μmol/L).

### Preparation of the Aggregate β-A₍₁₋₄₂₎

The β-A₍₁₋₄₂₎ was dissolved in a mixture of Acetonitrile and distilled water (CH₃CN/H₂O 1:1) to the final concentration of 1 mg/mL. An aliquot of 2 mL was freeze-dried to eliminate the trifluoroacetic acid residual of the peptide synthesis. The β-A₍₁₋₄₂₎ peptide was subsequently dissolved in 0.1 mL of DMSO and 5.0 mL of 2×PBS, pH 7.4. Once dissolved the β-A₍₁₋₄₂₎ was incubated to 37° C. for 8 days, at the end, after sonication, it was diluted five times with 2×PBS (final concentration 17.4 μmol/L). Waiting to be used, the aggregate β-A₍₁₋₄₂₎ was divided in aliquots and stored at −80° C.

Fluorescence Measurement with Thioflavin T

Scheme of added volumes in 96-well plates:

| | | | Aggregate | Test | Non aggregate |
|---|---|---|---|---|---|
| | PBS | H₂O | β-A₍₁₋₄₂₎ | compound | β-A₍₁₋₄₂₎ |
| Blank | 40 μL | 80 μL | — | — | — |
| Control sample | | 50 μL | 40 μL | — | 30 μL |
| Blank of test | 40 μL | 30 μL | — | 50 μL | — |
| compound | | | | | |
| Test compound | — | — | 40 μL | 50 μL | 30 μL |

The assay was performed in triplicate in 96-well plates as reported above in scheme. Test compounds were added in the wells containing the aggregate β-A₍₁₋₄₂₎ then, 15 after minutes, the non-aggregate β-A₍₁₋₄₂₎ was added. The 96-well plates were incubated at 37° C. under agitation for 24 hours.

The following day, a volume of 200 μL of a solution containing 10 μmol/L thioflavin T and 50 μmol/L Na₂HPO₄ pH 6.5 was added to each well. Fluorescence was measured in a VICTOR 2 (WALLAC) fluorescence spectrophotometer (λₑₓ=450 nm, λₑₘ=486 nm) (Findelis M. A et al).

Calculations and tables were elaborated by means of a PC.

The data were expressed as percent of residual aggregated β-A and, when possible, the dose reducing the aggregate formation of the 50% (IC₅₀) was estimate.

The % of aggregation was determinated by the following formula:

(βAmyloid+Test compound)−(Blank+Test compound)×100

(Control+βAmyloid)−Blank

### Results

Table A shows the IC₅₀ of the compounds. The results on compound ST1859 (1-[(2-hydroxy-1-naphthyl)methyl]-2-naphthol) (see WO02/00603) have been reported for comparative purposes.

TABLE A

| Compound | IC₅₀ (μM) |
|---|---|
| ST1859 | 23.5 |
| ST2177 | 1.0 |
| ST3458 | 2.2 |
| ST2762 | 2.4 |
| ST3459 | 2.6 |
| ST3451 | 2.7 |
| ST2761 | 5.70 |
| ST2178 | 5.13 |
| ST2175 | 5.59 |
| ST2176 | 5.43 |
| ST3501 | 7.2 |
| ST2757 | 14.1 |

### Example 9

### Blood Brain Barrier Crossing

In order to obtain basic information on the concentration achieved in brain of rodents after parental doses and their relationship with plasma concentrations, mice and rats were used. Animals were divided into groups and received compound subcutaneously or intravenously and were killed by decapitation 0, 15, 30, 60, 120, 180 and 240 min after dosing to determine plasma and brain concentrations of compounds. Compounds were determined in plasma by high-performance liquid chromatography (HPLC) after a solid liquid extraction procedure. Briefly, Oasis HLB 1 cc cartridges were pre-wetted with methanol and distilled water. Then internal standard, mouse plasma or rat plasma were added and the cartridges were washed with mater-methanoland methanol, interrupting the vacuum before the column was completely dry after each passage. The compound was removed by eluiting the cartridges with methanol and evaporated to dryness under nitrogen. The residue was dissolved in the mobile phase centrifuged and analyzed by HPLC with UV detection (224 nm).

Separation was done on a pBondapack C18 column protected by a LiChrosphere RP-8 pre-column at room temperature. The mobile phase was CH₃CN:CH₃OH:0.001M KH₂PO₄ (40:10:50 v/v) delivered at a flow rate of 1.2 mL/min.

Brain tissue was homogenized (1 g/10 ml) in CH₃CN:0.001M phosphate buffer, pH 7.4 and a volume containing approximately 100 mg of tissue was centrifuged. The supernatant was processed as for plasma.

Mean brain and plasma area under the concentration-time curve (AUCt) were determined using the linear trapezoidal rule and extrapolated to infinity (AUC) by the concentration method. The elimination rate constant was calculated by least squares regression analysis of the terminal log-linear portion of the plasma and the brain drug concentration curves. The maximum concentration (Cₘₐₓ) and the time (tₘₐₓ) of its occurrence were read directly from the plasma and brain concentration time data.

### Results

Table B show the plasma and brain concentration-time curves of compound ST2175 after s.c. injection (25 mg/kg) in mice.

TABLE B

| Time | Brain (ng/g) | Plasma (ng/ml) | Brain to Plasma |
|---|---|---|---|
| (min) | (mean ± SD) | (mean ± SD) | ratio |
| 0 | — | — | — |
| 15 | 321 ± 130 | 138 | 2.3 |
| 30 | 398 ± 269 | 109 | 3.6 |
| 60 | 410 ± 192 | 164 ± 18 | 2.5 |
| 120 | 289 ± 61 | 142 ± 16 | 2.0 |
| 180 | 253 ± 77 | 139 ± 31 | 1.8 |
| 240 | 220 ± 61 | 159 ± 38 | 1.3 |
| 360 | 232 ± 94 | 130 ± 13 | 1.7 |

Table C shows the plasma and brain AUC of compound ST2175 after s.c. injection (25 mg/kg) in mice.

TABLE C

| | | | Brain to Plasma |
|---|---|---|---|
| | Brain | Plasma | ratio |
| tₘₐₓ (min) | 60 | 60 | |
| Cₘₐₓ (ng/mL or L) | 410 | 164 | 2.5 |
| AUCt (ng/L · h or g) | 1643 | 850 | 1.9 |

1. A method of treating diseases characterised by deposits of amyloid aggregates comprising administering a compound of Formula (I) as a medicine 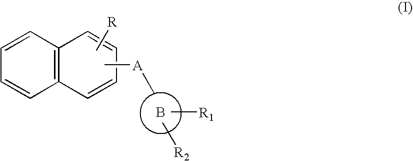
where:
R is selected from the group consisting of H, OR₃, COOR₃, N(R₃)₂, NO₂, halogen, hydroxyalkyl C₁-C₃;
R₁ and R₂ are the same or different and are selected from the group consisting of H; OR₃; COOR₃; linear or branched, saturated or unsaturated C₁-C₄ alkyl; N(R₃)₂; C₁-C₄ linear or branched, saturated or unsaturated alkylthio; halogen; and SO₂N(R₃)₂;
R₃ is selected from the group consisting of H; C₁-C₄ linear or branched alkyl; PO₃H₂ and PO₃(CH₃)₂;
A is selected from the group consisting of NR₄; S; and SO₂;
R₄ is selected from the group consisting of H; C₁-C₄ linear or branched alkyl; C₁-C₄ linear or branched alkanoyl; and
B is a phenyl or naphthyl group.

2. The method according to claim 1, wherein A is NH.

3. The method according to claim 1, wherein R₁ is H.

4. The method according to claim 1, wherein R₂ is selected from the group consisting of H, COOH, COOCH₃ and OH.

5. The method according to claim 1, wherein R is selected from the group consisting or H, OH and OCH₃.

6. The method according to claim 1, wherein the compound of Formula (I) is selected from the group consisting of:
1-hydroxy-N-phenylnaphthalen-2-aminium chloride;
methyl 4-(1-naphthylamino)benzoate;
4-(1-naphthylamino)benzoic acid;
4-(4-hydroxyanilino)-1-naphthol;
4-anilino-1-naphthol;
2-[(2-hydroxy-1-naphthyl)amino]benzoic acid;
(1-methoxy-2-naphthyl)phenylamine;
4-methoxy-N-phenyl-1-naphthalenamine;
1-methoxy-4-[(4-methoxyphenyl)sulfonyl]naphthalene; and
4-[(4-hydroxyphenyl)sulfonyl]-1-naphthol.

7. (canceled)

8. The method according to claim 1, in which the condition characterised by deposits of amyloid aggregates is selected from among the group consisting of Alzheimer's disease, Down's syndrome, hereditary cerebral haemorrhage accompanied by “Dutch type” amyloidosis, amyloidosis accompanied by chronic inflammation, amyloidosis accompanied by multiple myeloma and other dyscrasias of the haematic “B” lymphoid cells, amyloidosis accompanied by type II diabetes, amyloidosis accompanied by prion diseases, kuru or ovine scrapie.

9. The method according to claim 1, in which amyloidosis accompanied by prion diseases is selected from among the group consisting of Creutzfeldt-Jakob's disease or Gerstmann-Straussler syndrome.

10. A compound of Formula (I) 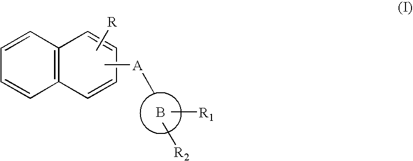
where:
R is selected from the group consisting of H, OR₃, COOR₃, N(R₃)₂, NO₂, halogen, hydroxyalkyl C₁-C₃;
R₁ and R₂ are the same or different and are selected from the group consisting of H; OR₃; COOR₃; linear or branched, saturated or unsaturated C₁-C₄ alkyl; N(R₃)₂; C₁-C₄ linear or branched, saturated or unsaturated alkylthio; halogen; and SO₂N(R₃)₂; provided that R₁ and R₂ are not both H or halogen;
R₃ is selected from the group consisting of H; C₁-C₄ linear or branched alkyl;
PO₃H₂; and PO₃(CH₃)₂;
A is selected from the group consisting of NR₄; S; and SO₂;
R₄ is selected from the group consisting of H; C₁-C₄ linear or branched alkyl;
C₁-C₄ linear or branched alkanoyl; and
B is a phenyl or naphthyl group,
with the proviso that:
when A is NR₄, R₁ and R₂ are not both OR₃; and
with the exception of the following compounds:
4-methoxy-N-phenyl-1-naphthalenamine;
1-hydroxy-N-phenylnaphthalen-2-aminium chloride;
methyl 4-(1-naphthylamino)benzoate;
4-(1-naphthylamino)benzoic acid,
4-(4-hydroxyanilino)-1-naphthol;
4-anilino-1-naphthol;
2-[(2-hydroxy-1-naphthyl)amino]benzoic acid;
(1-methoxy-2-naphthyl)phenylamine;
1-methoxy-4-[(4-methoxyphenyl)sulfonyl]naphthalene; and
4-[(4-hydroxyphenyl)sulfonyl]-1-naphthol.

11. The compound according to claim 10, wherein A is NH.

12. The compound according to claim 10, wherein R is selected between OH and OCH₃.

13. The compound according to claim 10 wherein R₁ is selected among OCH₃, COOCH₃, H and COOH.

14. The compound according to claim 10, wherein R₂ is selected among H, I, OH and OCH₃.

15. The compound according to claim 10, which is selected from the group consisting of:
methyl 2-[(2-methoxy-1-naphthyl)amino]benzoate;
1-methoxy-4-[(4-methoxyphenyl)thio]naphthalene;
N-(4-iodophenyl)-1-methoxynaphthalen-2-amine;
2-hydroxy-5-[(4-hydroxy-1-naphthyl)amino]benzoic acid;
methyl 2-[(2-hydroxy-1-naphthyl)amino]benzoate;
methyl 4-[(1-hydroxy-2-naphthyl)amino]benzoate
4-[(1-hydroxy-2-naphthyl)amino]benzoic acid;
4-[(1-methoxy-2-naphthyl)amino]benzoic acid;
methyl-4-[(1-methoxy-2-naphthyl)amino]benzoate;
4-[(4-hydroxy-1-naphthyl)amino]benzoic acid;
4-[(4-hydroxyphenyl)thio]-1-naphthol;
4-[(4-methoxy-1-naphthyl)amino]benzoic acid;
N,N-dimethyl-N′-[4-(methylthio)phenyl]naphthalene-1,4-diamine dihydrochloride;
4-fluoro-N-(4-fluorophenyl)naphthalen-1-amine hydrochloride;
4-fluoro-N-[4-(methylthio)phenyl]naphthalen-1-amine;
2-hydroxy-5-[(4-hydroxy-1-naphthyl)amino]benzoic acid hydrochloride;
4-methoxy-3-methylbenzoate-1-yl(4-methoxy-1-naphthyl)amine;
N-(5-iodo-2-methoxyphenyl)-N-(4-methoxy-1-naphthyl)amine;
N-(4-methoxy-1-naphthyl)-N-(2-methoxyphenyl)amine;
2-methoxy-5-[(4-methoxy-1-naphthyl)amino]benzoic acid;
4-methoxy-N-(4-methoxyphenyl)-1-naphthalenamine;
4-methylbenzoate-1-yl(4-methoxy-1-napthyl)amine;
N-(4-methoxyphenyl)-4-nitronaphthalen-1-amine
2-methoxy-N-(2-methoxy-1-naphthyl)naphthalen-1-amine; and
methyl 4-[(4-hydroxy-1-naphthyl)amino]benzoate.

16. A medicament containing a compound of claim 10.

17. (canceled)

18. A pharmaceutical composition containing as active ingredient a compound of claim 10, and at least one pharmaceutically acceptable excipient and/or diluent.

19. The pharmaceutical composition according to claim 18 for the treatment and/or prevention of disorders characterised by deposits of amyloid aggregates.

20. A process for preparing a compound of claim 1, comprising hydrogenating substituted or un-substituted nitro-naphthalene with catalyst in an organic solvent; condensating the resulting amine with a substituted or un-substituted aryl halide derivative in the presence of the reagent BINAP [2,2′-Bis(diphenylphosphino)-1,1′-binaphthyl] and palladium acetate.

21. (canceled)

22. Method of treating a mammal suffering from a disorder characterised by deposits of amyloid aggregates, comprising administering a therapeutically effective amount of a compound of claim 10.

23. A compound according to claim 10, in which at least one of the elements carbon, hydrogen, nitrogen or oxygen are replaced with a corresponding radioactive isotope.

24. The compound according to claim 23, containing at least one atom of radioactive iodine.

25. The compound according to claim 23, complexed with elements used in diagnostic imaging.

26. The compound of according to claim 25, in which the complexed element is selected from the group consisting of indium gadolinium or technetium.

27. A diagnostic kit, including at least one compound according to claim 10, for the diagnosis of diseases characterised by deposits of amyloid aggregates.

28. (canceled)

29. (canceled)

30. (canceled)
